(19) United States
(12) Patent Application Publication
(10) Pub. No.: US 2023/0404380 A1
(43) Pub. Date: Dec. 21, 2023

(54) ENDOSCOPIC AND/OR EXOSCOPIC IMAGING DEVICE FOR SPECTRAL IMAGING, AND METHOD FOR THE OPERATION THEREOF

(71) Applicant: KARL STORZ SE & Co. KG, Tuttlingen (DE)

(72) Inventors: Harald Baumann, Tuttlingen (DE); Johannes Fallert, Tuttlingen (DE); Werner Göbel, Tuttlingen (DE); Klaus Irion, Tuttlingen (DE)

(73) Assignee: KARL STORZ SE & Co. KG, Tuttlingen (DE)

(21) Appl. No.: 18/251,982

(22) PCT Filed: Nov. 8, 2021
(86) PCT No.: PCT/EP2021/080946

§ 371 Date: May 5, 2023

(30) Foreign Application Priority Data
Nov. 11, 2020 (DE) 10 2020 129 739.4

(51) Int. Cl. A61B 1/04; A61B 1/00

(52) U.S. Cl. CPC A61B 1/042; A61B 1/00126

(57) ABSTRACT

An endoscopic and/or exoscopic imaging device for spectral, in particular multispectral and/or hyperspectral, imaging for an endoscope, microscope and/or an exoscope, comprising at least one shaft and comprising at least one imaging channel situated at least in part in the shaft, which imaging channel comprises at least one first imaging branch that has having at least one first spectrally selective beam splitter that spectrally selectively splits an optical image of an original spectral range into at least one first partial spectral image of a first spectral range and at least one further first partial spectral image of a further first spectral range, wherein the first spectral range is different from the further first spectral range, and the first imaging branch comprises a first image sensor, at least for capturing the first partial spectral image, and the first imaging branch comprises at least one first relay lens for relaying the further first partial image.

### CROSS-REFERENCE TO RELATED APPLICATIONS

This application is the U.S. national stage of PCT/EP2021/080946 filed on Nov. 8, 2021, which claims priority of German Patent Application No. DE 2020 129 739.4 filed on Nov. 11, 2020, the contents of which are incorporated herein.

### TECHNICAL FIELD

The disclosure relates to an endoscopic and/or exoscopic imaging device.

### BACKGROUND

DE 10 2014 115 738 A1 already discloses an endoscopic imaging device for spectral imaging. Said device comprises a shaft and an imaging channel arranged in the shaft. The imaging channel has an imaging branch which comprises at least one first spectrally selective beam splitter which spectrally selectively splits an optical image representation into a spectral partial image representation of a spectral range and a further spectral partial image representation of a further spectral range. In that case, the spectral range is different from the further spectral range. The imaging branch comprises an image sensor, for capturing the spectral partial image representation. The imaging branch furthermore comprises a relay optical unit, for relaying the further partial image representation.

### SUMMARY

The object of the disclosure consists, in particular, in providing a device of the generic type having improved properties with regard to multispectral and in particular hyperspectral imaging, taking into account a compact design. The object is achieved according to the disclosure by means of the features of claim 1, while advantageous embodiments and developments of the disclosure can be gathered from the dependent claims.

The disclosure proceeds from an endoscopic and/or exoscopic imaging device for multispectral and in particular hyperspectral imaging of an examination region; comprising at least one shaft and comprising at least one imaging channel arranged at least partly, preferably at least largely and particularly preferably completely in the shaft and having at least one first imaging branch which comprises at least one first spectrally selective beam splitter which spectrally selectively splits an optical image representation of the examination region to be checked into at least one first spectral partial image representation of a first spectral range and at least one further first spectral partial image representation of a further first spectral range, wherein the first spectral range is different from the further first spectral range, and the first imaging branch comprises a first image sensor, at least for capturing the first spectral partial image representation, and the first imaging branch comprises at least one first relay optical unit, for relaying the further first partial image representation.

It is proposed that the imaging channel has at least one second imaging branch which comprises at least one second spectrally selective beam splitter which spectrally selectively splits the further first partial image representation into at least one second spectral partial image representation of a second spectral range and at least one further second spectral partial image representation of a further second spectral range, wherein the second spectral range is different from the further second spectral range, and the second imaging branch comprises at least one second image sensor, at least for capturing the second spectral partial image representation, and the second imaging branch comprises at least one second relay optical unit, for relaying the further second partial image representation, wherein the first relay optical unit of the first imaging branch and the second relay optical unit of the second imaging branch are arranged one behind the other such that an image plane of the first relay optical unit is identical to an object plane of the second relay optical unit.

As a result, a structural space of a multispectral and in particular hyperspectral endoscopic and/or exoscopic imaging device can advantageously be reduced. This is accomplished in particular by way of the arrangement of the image sensors of the different imaging branches. This is of interest precisely for the endoscopic use of the imaging device, since only the structural space that is absolutely necessary for a surgically minimally invasive intervention can be utilized in this application. As a further advantage, a spectral resolution can be increased, which can be multiplied by way of the number of imaging branches used. As a further advantage, the use of two imaging channels and the sensors thereof makes it possible to obtain a spectral representation in real time by means of a video, in particular by means of a stereo video.

An “endoscopic and/or exoscopic imaging device” should be understood to mean in particular a preferably functional constituent part, in particular a subassembly and/or a constructional and/or a functional component, of an endoscope and/or exoscope. The endoscopic and/or exoscopic imaging device can at least partly, preferably at least largely and particularly preferably completely, form the endoscope and/or the exoscope. The “endoscopic imaging device” is configured in particular to be inserted at least partly and preferably at least largely into an artificial cavity, such as, for example, a housing, and/or a natural cavity, such as, for example, a cavity in a body organ or in tissue, in order to assess same from inside. The “exoscopic imaging device” is configured in particular to be arranged outside an artificial cavity, such as, for example, a housing, and/or a natural cavity, such as, for example, a cavity in a body organ or in tissue, in order to assess same from outside. “Configured” should be understood to mean in particular specifically programmed, provided, embodied, designed and/or equipped. The fact that a component is configured for a specific function should be understood to mean in particular that the component fulfils and/or implements said specific function in at least one application state and/or operating state. In this case, the expression “at least largely” should be understood to mean in particular at least to the extent of 50%, with greater preference at least to the extent of 70%, preferably at least to the extent of 90%, and very particularly preferably completely, to be precise in particular in relation to a volume and/or a mass of a component. The endoscopic and/or exoscopic imaging device has at least one proximal portion and one distal portion. The distal portion is embodied in particular for being inserted in a cavity to be examined in an operating state. “Distal” should be understood to mean, during operator control, in particular, facing a patient and/or facing away from an operator. In particular, proximal is the opposite of distal. “Proximal” should be understood to mean, during operator control, in particular, facing away from a patient and/or facing an operator. The proximal portion is embodied in particular for being arranged outside the cavity to be examined in an operating state. The shaft can at least partly form the proximal portion, for example. The shaft is embodied in particular as an elongate component. The shaft is preferably embodied as a rigid shaft. Alternatively, the shaft could be embodied in a flexible fashion. An “elongate component” should be understood to mean in particular a component whose main extension is greater at least by a factor of five, preferably at least by a factor of ten, and particularly preferably at least by a factor of twenty, than a largest extension of the component perpendicular to the main extension thereof, i.e. in particular a diameter of the component. A “main extension” of a component should be understood to mean in particular the longest extension thereof along the main extension direction thereof. A “main extension direction” of a component should be understood to mean in particular a direction which runs parallel to a longest edge of a smallest imaginary parallelepiped which still just completely encloses the component.

“Spectral imaging” should be understood to mean in particular imaging which generates at least one spectral image representation of an examination region which comprises information of at least two spatial dimensions and at least one spectral dimension. The spectral image representation can thus be described by a data cube having at least row entries and column entries comprising spatial information and plane entries comprising spectral information. Furthermore, it is conceivable that the spectral image representation can also have a higher number of dimensions, such as, for example, in the form of a data hypercube or data tesseract, and can comprise three spatial dimensions, one temporal dimension and one spectral dimension. In this case, the spectral image representation would be present in the form of a five-dimensional data hypercube. The spectral information of a spectral image representation is available in particular on the basis of spectral characteristic values. On the basis of the spectral characteristic values, at least one type and/or at least one property, such as, for example, a tissue type and/or a tissue property, of the examination region to be observed can be deduced by means of the imaging device. The spectral characteristic values serve in particular as support points that can be compared with stored characteristic values that are characteristic of a specific type and/or property of the examination region in order to determine the at least one type and/or the at least one property of the examination region. “Multispectral imaging” should be understood to mean spectral imaging which captures a number of at least three, preferably at least five, and particularly preferably at least nine, spectral characteristic values which are spectrally offset with respect to one another. “Hyperspectral imaging” should be understood to mean in particular spectral imaging which captures a number of at least 10, preferably at least 40, and particularly preferably at least 120, spectral characteristic values which are spectrally offset with respect to one another. In this case, the captured spectral characteristic values lie in particular in a wavelength range starting from at least nm, preferably starting from at least 150 nm, and particularly preferably starting from at least 300 nm, and/or up to at most 3000 nm, preferably up to at most 2000 nm and particularly preferably up to at most 1000 nm, i.e. for example also within the ultraviolet and infrared wavelength range.

An “imaging channel” should be understood to mean in particular an optical transmission path which contributes to capturing the spectral image representation. An imaging branch of an imaging channel is in particular a portion of the imaging channel along which the latter has a branching of an optical transmission path or beam path. A “relay optical unit” of an imaging branch should be understood to mean in particular an optical unit which lengthens an image representation along an optical transmission path and inverts an intermediate image of the image representation at least once inverts along the transmission path. The relay optical unit of the imaging branch can comprise one or more lenses, preferably at least one gradient lens, and achromats, apochromats or the like, for example for correcting chromatic imaging aberrations.

A spectrally selective beam splitter is in particular an optical component which splits an optical image representation into at least one first spectral partial image representation of a first spectral range and at least one further first spectral partial image representation of a further first spectral range, although the spatial information of the image representation is also maintained in the respective partial image representations. The beam splitter can be embodied as a reflection beam splitter, interference beam splitter, beam splitter cube, dichroic mirror or the like. The fact that “one spectral range is different from a further spectral range” should be understood to mean in particular that the spectral ranges spectrally at least partly, preferably at least largely, and particularly preferably completely, do not mutually overlap. The spectral ranges can have in particular different spectral widths. The first spectral partial range preferably extends over the visible spectral range. The second spectral range particularly preferably extends over the near-infrared spectral range.

An “image sensor” should be understood to mean in particular a sensor which has sensor pixels arranged in a two-dimensional matrix. The image sensor can be a CMOS sensor, a CCD sensor or the like. The image sensor is configured in particular to record images with a frame rate of at least 15, preferably at least 30, and particularly preferably at least 60, frames per second, whereby spectral information can be recorded and/or reproduced as video or in real time. The image sensor has in particular at least one spectral sensitivity which defines the wavelength-dependent sensitivity of the image sensor and which is describable by a sensitivity characteristic curve. The spectral sensitivity is in particular dependent on a quantum efficiency of a sensor pixel of the image sensor and/or of a filter spectrum of a sensor filter of the image sensor, said filter spectrum being assigned to the sensor pixel. A spectral sensitivity can thus be varied by the use of different sensor filters or sensor pixels. If for example light of a partial image representation of a spectral range of this partial image representation passes to the image sensor, then the image sensor registers this depending on the respective spectral range of the partial image representation and the spectral sensitivity of the image sensor itself. A spectral characteristic value sought results from the superposition of the transmitted light of the partial image representation of the spectral range of the partial image representation and the spectral sensitivity of the sensor.

As described above, a number of support points that can be determined by the imaging device can be scaled with the number of different spectrally selective beam splitters of the imaging branches. In order advantageously to further increase a spectral resolution and nevertheless to maintain a compact design, it is proposed that the imaging channel has at least one third imaging branch which comprises at least one third spectrally selective beam splitter which spectrally selectively splits the further second partial image representation into at least one third spectral partial image representation of a third spectral range and at least one further third spectral partial image representation of a further third spectral range, wherein the third spectral range is different from the further third spectral range, and the third imaging branch comprises at least one third image sensor, at least for capturing the third spectral partial image representation, and the third imaging branch comprises at least one third relay optical unit, for relaying the further third partial image representation, wherein the second relay optical unit of the second imaging branch and the third relay optical unit of the third imaging branch are arranged one behind the other such that an image plane of the second relay optical unit is identical to an object plane of the third relay optical unit. The third spectral range preferably extends over the infrared spectral range. Particularly advantageously, the spectral resolution can be increased further by a multiplicity of imaging branches being arranged one behind another in a cascaded manner in accordance with the arrangement of the first and second imaging channels.

In order advantageously to further reduce a structural space and to obtain a space-saving arrangement, it is furthermore proposed that at least one spectrally selective beam splitter and/or at least one image sensor of the imaging branches are/is arranged between two relay optical units of different imaging branches. In particular, the at least one spectrally selective beam splitter and/or the at least one image sensor of the imaging branches are/is arranged between a relay optical unit of one of the imaging branches and the image plane thereof. Furthermore, preferably in each case at least the one image sensor is assigned to the at least one beam splitter of the imaging branch and is disposed optically downstream thereof. It is conceivable that further optical components, such as, for example, lenses, prisms or the like, can be arranged between the beam splitter and the image sensor assigned to the beam splitter. Advantageously, the image sensor is arranged in a manner directly succeeding the beam splitter. In particular, the image sensor could be connected to the beam splitter in a force-locking and/or positively locking manner. “In a force-locking and/or positively locking manner” should be understood to mean in particular that a holding force between two components is preferably transmitted by a geometric engagement of the components into one another and/or a frictional force between the components. Furthermore, the image sensor and the beam splitter could also be cohesively connected to one another. Furthermore, they could also be integrally connected to one another. “Integrally” should be understood to mean in particular at least cohesively connected, for example by way of a welding process, an adhesive bonding process, and injection process and/or any other process that appears to be expedient to a person skilled in the art, and/or should be understood to mean advantageously shaped in one piece, such as, for example, by way of production from a casting and/or by way of production in a single- or multi-component injection molding method and advantageously from a single blank. Preferably, respective spectrally selective beam splitters and/or image sensors of adjacent imaging branches are arranged between two relay optical units of the adjacent imaging branches. Particularly preferably, this holds true for all beam splitters and/or image sensors of the imaging branches arranged in a cascade-like manner.

Furthermore, it is proposed that at least one of the imaging branches comprises at least one further optical component, the refractive index of which and/or the material of which at least substantially correspond(s) to a refractive index and/or a material of a spectrally selective beam splitter of the respective imaging branch, and is arranged between two relay optical units of different imaging branches. Advantageously, chromatic imaging aberrations that would otherwise reduce an accuracy of a spectral analysis and also of an optical image representation can be compensated for or avoided. As a further advantage, a required structural space can be minimized despite additional components. The expression “at least substantially” with regard to a property, a numerical value or the like should be understood to mean in particular the property, the numerical value or the like, to be precise preferably taking into account a deviation of at most 15%, preferably of at most 10%, and particularly preferably of at most 5%. In particular, the at least one further optical component of the imaging branches is arranged between a relay optical unit of one of the imaging branches and the object plane thereof. The further optical component is preferably embodied as a glass cube. Preferably, each of the imaging branches has a corresponding further optical component, which is also correspondingly arranged between adjacent imaging branches.

In order to avoid imaging aberrations by way of a symmetrical arrangement, it is additionally proposed that the spectrally selective beam splitter and the further optical component are arranged mirror-symmetrically with respect to the mutually identical image plane and object plane of two, in particular adjacent, relay optical units of different imaging branches. Preferably, respective spectrally selective beam splitters and respective further optical components are arranged mirror-symmetrically with respect to the mutually identical image plane and object plane of two relay optical units of adjacent imaging branches.

Furthermore, it is proposed that the spectrally selective beam splitter and the further optical component are embodied with or connected to one another at least partly integrally. The generation of further imaging aberrations in boundary regions between these components can advantageously be avoided. As a further advantage, reflections or scatterings can be avoided.

It is additionally proposed that at least one of the spectral ranges of the partial image representations of the imaging branches comprises shorter wavelengths than at least one other spectral range of the partial image representations of the imaging branches. A spectral splitting of the image representation into the partial image representations can advantageously be improved. Particularly preferably, all the spectral ranges of the partial image representations are different from one another. In particular, the wavelengths of the spectral ranges of the partial image representations increase in a proximal or distal direction in a stepwise manner for each of the imaging branches arranged one behind another.

Furthermore, it is proposed that at least one image sensor or at least two image sensors of the imaging branches has/have a sensor plane arranged at least substantially parallel to a central axis of the shaft. A particularly compact arrangement of the image sensors can advantageously be obtained. A “sensor plane” should be understood to mean in particular a main extension plane of the sensor. In the sensor plane, in particular the sensor pixels are arranged in a matrix. Preferably, all the sensor planes of all the image sensors of all the imaging branches are arranged at least substantially parallel to the central axis. Particularly preferably, all the image sensors lie in a single plane lying at least substantially parallel to the central axis of the shaft. “Substantially parallel” should be understood here to mean in particular an orientation of a direction relative to a reference direction, in particular in a plane, wherein the direction has a deviation of in particular less than 8°, advantageously less than 5°, and particularly advantageously less than 2°, relative to the reference direction.

It is additionally proposed that the imaging channel has at least one printed circuit board on which at least one image sensor or at least two image sensors of the imaging branches, and particularly preferably all of the image sensors of the imaging branches, is/are arranged. A space-saving and simple arrangement of the image sensors for the purpose of making electrical contact therewith can advantageously be obtained. The printed circuit board defines in particular a plane lying at least substantially parallel to the central axis of the shaft. The printed circuit board could be embodied as a flexible printed circuit. Preferably, the printed circuit board is embodied as a PCB. The printed circuit board is arranged in particular in the shaft. Furthermore, it is conceivable for the printed circuit board to be integrated into the shaft.

It is proposed that at least one image sensor or at least two image sensors of the imaging branches has/have at least one first spectral sensitivity and at least one second spectral sensitivity, and in particular at least one third spectral sensitivity, depending on which said sensor(s) capture(s) a partial image representation of the imaging branches. Advantageously, a spectral resolution can be increased further. The latter can be increased further by way of the number of different spectral sensitivities of the image sensors. The fact that an image sensor has different spectral sensitivities can be obtained by the sensor pixels being assigned to different sensor filters. The image sensor can be embodied for example as an RGB sensor comprising a first spectral sensitivity in the blue visible spectrum, a second spectral sensitivity in the green visible spectrum and a third spectral sensitivity in the red visible spectrum.

It is conceivable for all the image sensors of the imaging branches to have identical spectral sensitivities. However, a spectral resolution of the imaging device can be increased further with a number of different spectral sensitivities of the different image sensors. It is therefore proposed that at least one image sensor of the imaging branches has at least one spectral sensitivity which is different from a spectral sensitivity of another image sensor of the imaging branches. Preferably, the spectral sensitivities of all the image sensors are different from one another.

It is proposed that the imaging channel has at least one camera arranged at a proximal and in particular extracorporeal end portion of the shaft, for capturing at least one of the partial image representations of the imaging branches. Advantageously, additional image properties can be captured by means of the separate camera. An “end portion” should be understood to mean in particular a portion which proceeds from one end of a component and extends in the direction of the center of the component. In order to particularly increase a spectral resolution, it is proposed that the camera is embodied as a multispectral and/or hyperspectral camera. Particularly preferably, the camera operates according to the spatial scanning principle, but other imaging principles known to a person skilled in the art are also conceivable.

It is further proposed that the imaging device comprises at least one light source which illuminates an examination region to be imaged with at least one illumination spectrum in at least one operating state. Since the illumination spectrum is known, this can be included in an evaluation, as a result of which an information content of the evaluation can be improved. The light from the light source impinges in particular on the examination region to be imaged and is at least partly absorbed, transmitted or reflected there. The absorbed portion of the light from the light source can be re-emitted by the examination region at least partly in the form of fluorescence and/or phosphorescence. By means of the imaging device, at least one reflective portion of the light from the light source and/or fluorescence and/or phosphorescence emitted by the examination region can be utilized for the imaging of the examination region. Moreover, it is conceivable to capture the transmitted portion by means of the present imaging device. The light source can be a broadband light source, a white-light light source, a laser light source or the like. The light source can have at least one and preferably a plurality of light elements, such as, for example, LEDs, OLEDs, lasers or the like. The illumination spectrum of the light source could be variable over time, e.g. by way of turning off and/or supplementarily turning on different light elements. The light source comprises at least one light element which generates white light. “White light” should be understood to mean in particular polychromatic light which at least substantially corresponds to the daylight spectrum at least in the visible spectral range. The visible spectral range in this case extends in particular between 380 nm and 780 nm.

It is proposed that the light source emits illumination light comprising an illumination spectrum which is broader than at least one spectral range of the partial image representations of the imaging branches. That range of the spectrum which is of interest for the spectral examination can advantageously be covered by one light source. The light source can emit in particular at least two and preferably a plurality of illumination lights which are different from one another. These can differ in terms of their spectral emission behavior. The light source has in particular at least one light source element. Preferably, the light source comprises at least two and particularly preferably a plurality of light source elements. The light source element can be an LED, OLED, laser diode or the like. The light source elements can differ from one another in terms of an illumination light provided by them.

Alternatively or additionally, it is proposed that the light source emits illumination light comprising at least one illumination spectrum which lies within at least one spectral range of the partial image representations of the imaging branches. A particularly sharp spectral resolution can advantageously be obtained.

It is furthermore proposed that the imaging device comprises at least one further imaging channel. An information content of the spectral imaging can advantageously be increased since in particular by way of the combination of the information of the imaging channel and of the further imaging channel, stereoscopic information can also be obtained in addition to the spectral information. The imaging channel and the further imaging channel differ at least in terms of a segment—which was registered by means of the respective imaging channel—of an examination region by means of the endoscopic and/or exoscopic imaging device. The imaging channel and the further imaging channel are arranged in particular offset with respect to one another, to be precise preferably at least substantially perpendicular to central axes of the respective imaging channels.

Furthermore, the imaging channel and the further training path can have different viewing directions; by way of example, the imaging channels can be arranged in click with respect to one another. Preferably, however, the imaging channel and the further imaging channel are arranged at least substantially parallel to one another. In particular, the imaging device could have a plurality of imaging channels which could differ from one another at least in terms of a viewing direction and/or could be arranged offset with respect to one another.

It is additionally proposed that the imaging channels differ from one another at least in terms of a spectral sensitivity of at least one spectrally selective beam splitter of their imaging branches and/or in terms of a spectral sensitivity of at least one image sensor of their imaging branches. A spectral resolution can advantageously be increased further. The images and spectral information obtained by way of the different imaging channels, a stereoscopic and spectrally resolved image can be generated. In particular, the spectral information can be matched to the stereoscopic information.

It is proposed that the imaging channel and the further imaging channel are arranged mirror-symmetrically with respect to one another. A particularly structurally compact arrangement of the imaging channels with respect to one another can advantageously be obtained. In particular, the image sensors of the imaging channel and of the further imaging channel are arranged on the same printed circuit board, to be precise preferably mirror-symmetrically with respect to one another.

In a further aspect of the disclosure, which can be considered by itself but also in combination with the preceding aspect, it is proposed that the imaging device at least has at least one further imaging channel, which is free of a relay optical unit. A stereo imaging device which is suitable even for endoscopes having small shaft diameters can advantageously be provided as a result. In particular, the further imaging channel comprises at least one further objective lens and an image sensor arranged so as to follow the objective lens downstream in terms of luminous flux. Furthermore, the further imaging branch can have a mirror and/or a beam splitter, which at least partly, preferably at least largely, and particularly preferably completely, deflects light entering through the objective lens. The beam splitter is in particular a spectrally selective beam splitter.

Furthermore, it is proposed that the shaft is substantially filled by the imaging channel at least in portions. A shaft diameter can advantageous be reduced since it can be adapted just to the dimensions of the imaging channel and not of the further imaging channel. The fact that the shaft is filled by the imaging channel in portions should be understood to mean in particular that the shaft has at least one portion, for example a central portion, between distal and proximal end portions in which the shaft is filled by the imaging channel at least to the extent of 50%, preferably at least to the extent of 65%, and particularly preferably at least to the extent of 80%, of its volume taken up by the imaging channel.

In order that different imaging methods can be utilized simultaneously, it is furthermore proposed that the imaging channel is configured for multispectral and/or hyperspectral imaging and the further imaging channel is configured for white light imaging.

Furthermore, it is proposed that the imaging device comprises a control device configured to match at least one multispectral and/or hyperspectral image recorded by the imaging channel with at least one white light image recorded by the further imaging channel. Multispectral and/or hyperspectral stereo imaging can be obtained digitally as a result. Such matching of the white light image and the multispectral and/or hyperspectral image can be effected by means of a matching algorithm stored in the control device. Said algorithm can be based for example on the methodology of image stabilization, feature tracking, marker tracking or the like.

Furthermore, it is proposed that at least one temperature sensor configured for temperature-dependent control of at least the imaging channel is arranged in a distal end portion of the shaft. Advantageously, a temperature increase produced by distal electronics can be monitored and a limit temperature that could lead to injuries to a patient and/or damage to the imaging device can be prevented from being exceeded by targeted activation and/or deactivation of the electronics, such as the image sensors, for example. Furthermore, the temperature sensor can alternatively or additionally be configured for the temperature-dependent control of the further imaging channel. For this purpose, the temperature sensor is more or less coupled to the control device. In particular, the limit temperature to be complied with also determines an irradiation time to be complied with for the light source.

It is further proposed that at least one motion sensor configured for motion-dependent control of at least the imaging channel is arranged in a distal end portion of the shaft. The motion sensor is configured to register a constant position of the imaging device during a recording of an image representation. Particularly for the case where a motion is registered, a message is transferred to the control device and a recording is stopped. Furthermore, it is conceivable for a motion to be recorded by means of the motion sensor and for imaging to be corrected on the basis of this detected motion.

Furthermore, an endoscope and/or exoscope comprising at least one such endoscopic and/or exoscopic imaging device is proposed. A structural space of a multispectral and in particular hyperspectral endoscopic and/or exoscopic imaging device can advantageously be reduced as a result.

Furthermore, a method for operating such an imaging device is proposed. As a result, it is possible to provide a hyperspectral endoscopic and/or exoscopic imaging function for devices with a small structural space.

In this case, the imaging device, the endoscope and/or exoscope and/or the operating method shall not be restricted to the application and embodiment described above. In particular, the imaging device, the endoscope and/or exoscope and/or the operating method, in order that functioning described herein is fulfilled, can have a number of individual elements, components and units and also method steps that deviates from a number mentioned herein. Moreover, in the case of the value ranges specified in this disclosure, values lying within the limits mentioned shall also be deemed to be disclosed and to be usable as desired.

It is pointed out, in particular, that all features and properties but also procedures described with regard to the device are also analogously applicable to the method according to the disclosure and usable within the meaning of the disclosure and are deemed to be concomitantly disclosed. The same applies conversely as well. That means that structural, i.e. device-pertaining, features mentioned with regard to the method can also be taken into account, claimed and likewise included as part of the disclosure within the scope of the device claims.

If there is more than one instance of a specific component, only one of them is provided with a reference sign in the figures and in the description. The description of this instance can accordingly be applied to the other instances of the component.

### BRIEF DESCRIPTION OF DRAWINGS

Further advantages will become apparent from the following description of the drawings. The drawings illustrate an exemplary embodiment of the disclosure.

The drawings, the description and the claims contain numerous features in combination. A person skilled in the art will expediently also consider the features individually and combine them to form expedient further combinations.

In the figures:

FIG. 1 shows a schematic illustration of an imaging device in a perspective view,

FIG. 2 shows a schematic illustration of a part of the imaging device in a sectional view,

FIG. 3 shows a set-up of a camera of the imaging device in a schematic illustration,

FIG. 4 shows a schematic diagram illustrating the spectral properties of a light source of the imaging device,

FIG. 5 shows a schematic diagram illustrating exemplary spectral properties of an examination region to be imaged by means of the imaging device,

FIG. 6 shows a schematic diagram illustrating optical properties of beam splitters of the imaging device in an idealized manner,

FIG. 7 shows a schematic diagram illustrating further optical properties of beam splitters of the imaging device in an idealized manner,

FIG. 8 shows a schematic diagram illustrating detection properties of the image sensors of the imaging device,

FIG. 9 shows a schematic diagram illustrating spectral sensitivities of the image sensors,

FIG. 10 shows a schematic diagram illustrating spectral sensitivities of the image sensors,

FIG. 11 shows a schematic diagram illustrating spectral sensitivities of the image sensors,

FIG. 12 shows a schematic flow chart of an exemplary method for operating the endoscopic imaging device,

FIG. 13 shows a schematic illustration of an exoscopic imaging device in a perspective view,

FIG. 14 shows a schematic diagram with optical properties of an alternative imaging device,

FIG. 15 shows a schematic diagram with optical properties of a further embodiment of a further endoscopic and/or exoscopic imaging device,

FIG. 16 shows a schematic diagram of an alternative endoscopic and/or exoscopic imaging device in a sectional view,

FIG. 17 shows a schematic illustration of an alternative endoscopic and/or exoscopic imaging device in a sectional view,

FIG. 18 shows a schematic illustration of a further alternative endoscopic and/or exoscopic imaging device in a sectional view, and

FIG. 19 shows a schematic illustration of a spectral splitting of an optical image representation recorded by the imaging device into partial image representations.

### DETAILED DESCRIPTION OF PREFERRED EMBODIMENTS

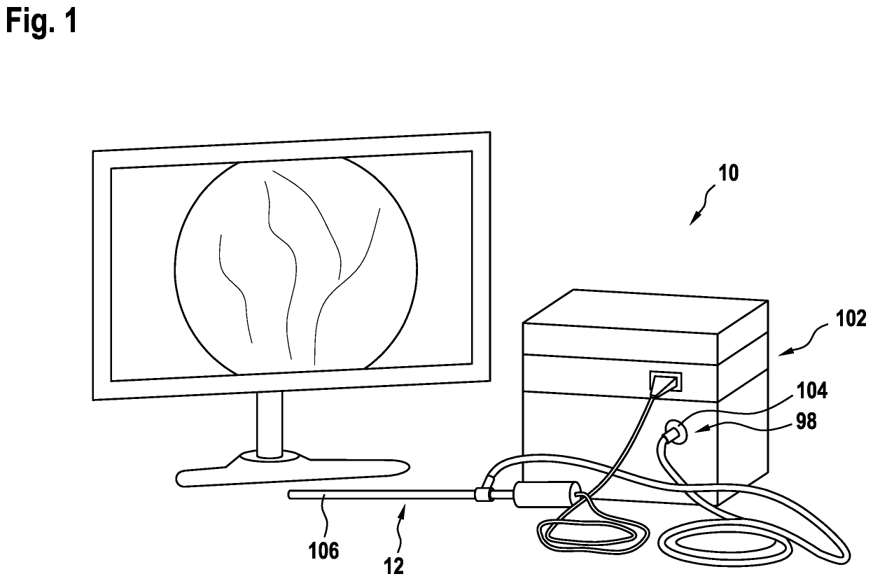

FIG. 1 shows a schematic illustration of an endoscopic imaging device 10 in a perspective view. The endoscopic imaging device 10 completely forms an endoscope 12. Alternatively, the endoscopic imaging device could form only a functional constituent part of an endoscope. Alternatively or additionally, the imaging device described herein can also be an exoscopic imaging device. Such an exoscopic imaging device could completely form an exoscope or could form only a functional constituent part of the exoscope. The suitability of the same device for both the exoscopic and the endoscopic use is also conceivable. In particular, the following description is also applicable to an exoscopic imaging device 10.

The endoscopic imaging device 10 is configured for spectral imaging. In the present case, the endoscopic imaging device 10 is configured at least for multispectral imaging. Furthermore, the endoscopic imaging device 10 can even be configured for hyperspectral imaging. The endoscopic imaging device 10 is configured to image at least one examination region in an operating state. In the present case, the examination region is tissue, for example of a body part. The examination region can lie within a body. In this respect, the endoscopic imaging device 10 is configured to be arranged at least partly in a cavity.

The imaging device 10 has a control device 102. The control device 102 is configured to control further functional components of the endoscopic imaging device 10. The control device 102 has control electronics (not illustrated). The control electronics comprise a processor. Furthermore, the control electronics have a memory. A program for operating the endoscopic imaging device 10 is stored on the memory. The processor is configured to execute the program for operating the endoscopic imaging device 10. In the present case, the control device 102 is arranged in a common housing with further components of the endoscopic imaging device 10. Alternatively, however, the control device could be embodied as a separate control unit.

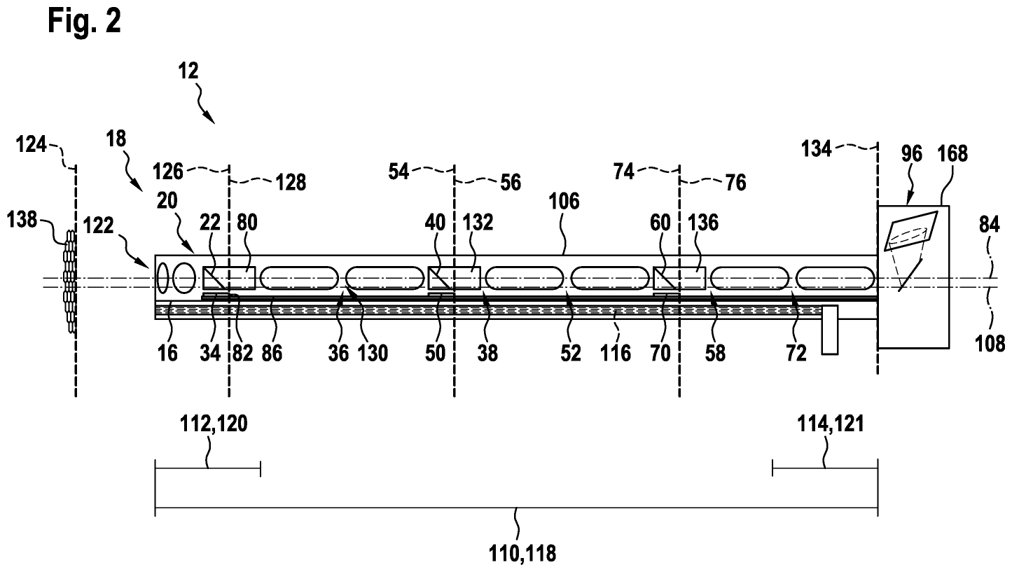

The endoscopic imaging device 10 has an overshaft 106. The overshaft 106 is embodied as an elongate component. The overshaft 106 has a central axis 108 (cf. FIG. 2). A main extension 110 of the overshaft 106 runs along the central axis 108. A cross section of the overshaft 106 perpendicular to the central axis 108 thereof has a diameter that is significantly less than the main extension 110 of the overshaft 106. The diameter is less than the main extension 110 at least by a factor of 2. In the present case, the diameter is less than the main extension 110 even at least by a factor of 5. The overshaft 106 is embodied in tubular fashion. The overshaft 106 is formed from metal. By way of example, the overshaft can be formed from steel, titanium, chromium or the like. The overshaft 106 has a distal end portion 112. Furthermore, the overshaft 106 has a proximal end portion 114. The overshaft 106 is configured to be inserted into a cavity via a naturally or artificially constituted opening, such as a body orifice, for example, in an operating state.

FIG. 2 shows a schematic illustration of a part of the endoscopic imaging device 10 in a sectional view. The endoscopic imaging device 10 has at least one shaft 16. The shaft 16 is arranged in the overshaft 106. In the present case, the shaft 16 is embodied as an elongate component. The shaft 16 has a central axis 84. A main extension 118 of the shaft 16 runs along the central axis 84. A cross section of the shaft 16 perpendicular to the central axis 84 thereof has a diameter that is significantly less than the main extension 118 of the shaft 16. The diameter is less than the main extension 118 at least by a factor of 2. In the present case, the diameter is less than the main extension 118 even at least by a factor of 5. The shaft 16 is embodied in tubular fashion. The shaft 16 is formed from metal. By way of example, the shaft 16 can be formed from steel, titanium, chromium or the like. The shaft 16 has a distal end portion 120. Furthermore, the shaft 16 has a proximal end portion 121.

The endoscopic imaging device 10 has at least one imaging channel 18. The imaging channel 18 is at least partly arranged in the shaft 16. The imaging channel 18 is configured to transmit an optical image representation 24 of an examination region 138 to be imaged at least partly along the shaft 16.

The imaging channel 18 has an objective lens 122. The objective lens 122 is arranged at least partly in the shaft 16. The objective lens 122 is arranged in the distal end portion 120 of the shaft 16. The objective lens 122 is configured for generating an optical image representation 24 of the examination region. The objective lens 122 has an object plane 124. In an operating state, the examination region is situated in the object plane 124. The objective lens 122 furthermore has an image plane 126. The objective lens 122 images an optical image representation 24 of the examination region situated in the object plane 124 onto the image plane 126.

The imaging channel 18 has a first imaging branch 20. The first imaging branch 20 is arranged at least partly in the shaft 16. In the present case, the first imaging branch 20 is arranged completely in the shaft 16. The first imaging branch is disposed after the objective lens 122 upstream in terms of luminous flux.

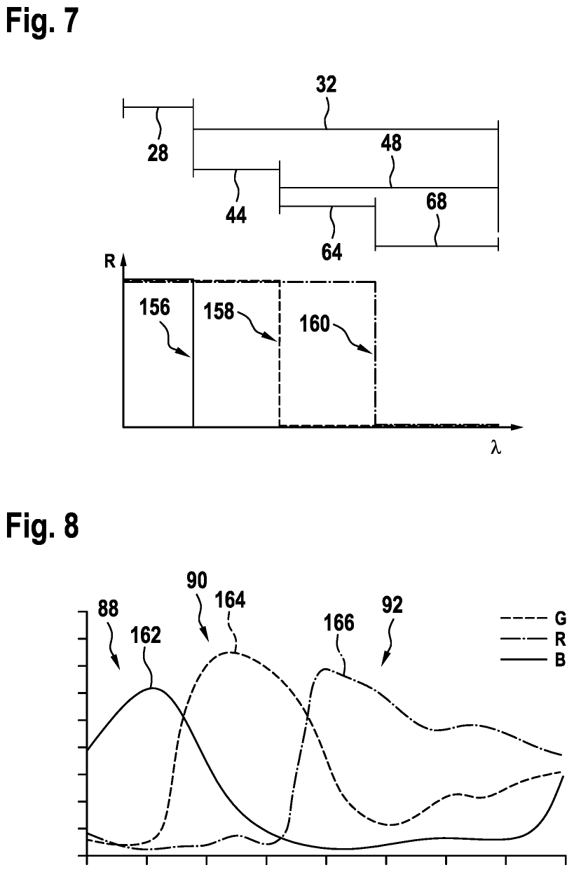

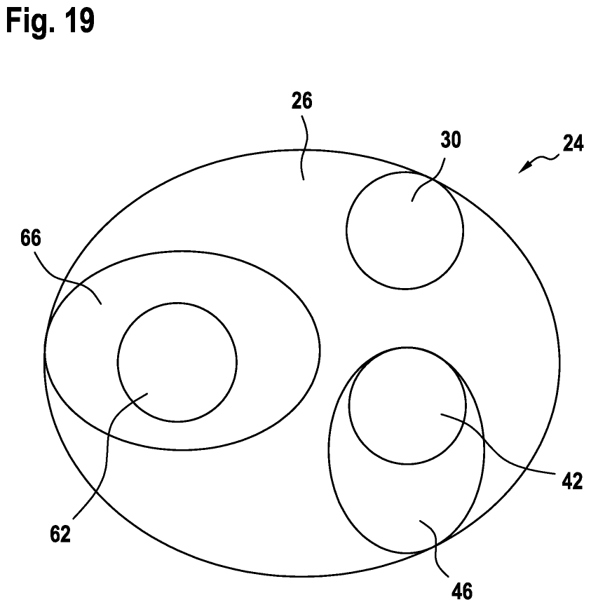

The first imaging branch 20 has at least one first spectrally selective beam splitter 22. The first spectrally selective beam splitter 22 splits the optical image representation 24 of the examination region into at least one first spectral partial image representation 26 of a first spectral range 28 and at least one further first spectral partial image representation 30 of a further first spectral range 32. The partial image representations 26, 30 and the spectral ranges 28, 32 are illustrated in FIGS. 6 and 7, which will be described in even greater detail below. The optical image representation 24 and also the partial image representations are illustrated in FIG. 19. The first spectral range 28 is different from the further first spectral range 32. The first spectrally selective beam splitter 22 deflects the first spectral partial image representation 26, by 90° in the present case. The further first spectral partial image representation 30 passes through the first spectral beam splitter 22 in a constant direction. It is conceivable that the spectral ranges could be variable by way of control of the beam splitter. By way of example, an angle of a reflection plane of the beam splitter relative to the incident light beam or to the central axis 84 could be varied in order to achieve this. In the present case, however, an angle of the reflection plane of the first spectral beam splitter 22 is chosen to be fixed. The angle is 45° with respect to the incident light beam or with respect to the central axis 84 of the shaft 16.

The first imaging branch 20 has at least one first image sensor 34. The first image sensor 34 is configured for capturing the first spectral partial image representation 26. The first spectral partial image representation 26 is directed onto the first image sensor 34 as a result of the deflection of the first spectrally selective beam splitter 22. The first image sensor 34 lies in an image plane of the first spectral partial image representation 26. Consequently, the first spectral partial image representation 26 is sharply imaged onto the first image sensor 34. The first image sensor 34 has a sensor plane 82. The sensor plane 82 is arranged at least substantially parallel to the central axis 84 of the shaft 16.

For the purpose of relaying the further first partial image representation the first imaging branch 20 has at least one first relay optical unit 36. The first relay optical unit 36 is disposed after the first selective beam splitter 22 upstream in terms of luminous flux. The first relay optical unit 36 images the further first partial image representation 30 from an object plane 128 of the first relay optical unit into an image plane 54. The objective lens 122 and the first relay optical unit 36 are arranged one behind the other such that the image plane 126 of the objective lens 122 is identical to the object plane 128 of the first relay optical unit 36. In this way, the first relay optical unit 36 relays the further first partial image representation 30 true to scale. The first relay optical unit 36 has a rod lens pair 130. Furthermore, the first relay optical unit 36 has two achromats, apochromats or the like (not illustrated). These can be arranged between the rod lens pair. These make it possible to reduce chromatic imaging aberrations that intensify further owing to the cascade-like arrangement of a plurality of imaging branches as described below. Furthermore, other embodiments of the relay optical unit are also conceivable. By way of example, the relay optical unit could also just consist of one rod lens, for example a gradient lens.

The first spectrally selective beam splitter 22 is arranged between the objective lens 122 and the first relay optical unit 36 of the first imaging branch 20. To put it more precisely, the first spectrally selective beam splitter 22 is arranged between the objective lens 122 and the image plane 126 of the objective lens 122 or the object plane 128 of the first relay optical unit 36.

The first image sensor 34 is arranged between the objective lens 122 and the first relay optical unit 36 of the first imaging branch 20. To put it more precisely, the first image sensor 34 is arranged between the objective lens 122 and the image plane 126 of the objective lens 122 or the object plane 128 of the first relay optical unit 36. The first image sensor 34 is disposed after the first spectrally selective beam splitter 22 upstream in terms of luminous flux. In this case, the first spectrally selective beam splitter 22 and the first image sensor 34 are arranged such that an optical distance through the first spectrally selective beam splitter 22 to the first image sensor 34 corresponds identically to an optical distance through the first spectrally selective beam splitter 22 to the image plane 126 of the objective lens 122 or the object plane 128 of the first relay optical unit 36.

The first imaging branch 20 has at least one first further optical component 80. A refractive index and and/or a material of the first further optical component 80 at least substantially corresponds to a refractive index and/or a material of the first spectrally selective beam splitter 22. The first further optical component 80 is arranged between the objective lens 122 and the first relay optical unit 36. To put it more precisely, the first further optical component 80 is arranged between the first spectrally selective beam splitter 22 and the first relay optical unit 36. The further optical component 80 is arranged in such a way that the image plane 126 of the objective lens 122 or the object plane 128 of the first relay optical unit 36 lies between the first further optical component 80 and the first spectrally selective beam splitter 22. The first spectrally selective beam splitter 22 and the first further optical component 80 are arranged mirror-symmetrically with respect to the mutually identical image plane 126 of the objective lens 122 and the object plane 128 of the first relay optical unit 36. In the present case, the first further optical component 80 is integrally connected to or embodied with the first spectrally selective beam splitter 22. By way of example, the beam splitter and the further optical component could be adhesively bonded or cemented to one another. Furthermore, it is conceivable for the beam splitter and the further optical component to be at least partly manufactured integrally out of one blank. Alternatively, the further optical component and the beam splitter could also be embodied separately from one another.

In the present case, the imaging channel 18 has a plurality of imaging branches 20, 36, 58, specifically for example in the present case a first imaging branch 20, a second imaging branch 38 and a third imaging branch 58. In the present case, the imaging branches 20, 38, 58 are embodied substantially identically apart from their spectral properties. Therefore, only the specific arrangement of the imaging branches 20, 38, 58 and the components thereof with respect to one another will be discussed hereinafter. Unless stated otherwise, a description of the components of the imaging branch 20 is applicable to components of the imaging branches 38, 58. Although the imaging channel 18 has a total of three imaging branches in this embodiment, it is conceivable that the number can be adapted, and in particular increased, in an obvious manner to a person skilled in the art in order to increase a number of spectral characteristic values that can be ascertained by means of the imaging branches 20, 38, 58. A spectral resolution of the device could be improved as a result.

The imaging channel 18 has at least one second imaging branch 38. The second imaging branch 38 is arranged at least partly in the shaft 16. In the present case, the second imaging branch 38 is arranged completely in the shaft 16. Furthermore, the second imaging branch 38 is disposed after the first imaging branch 20 upstream in terms of luminous flux. Furthermore, the first imaging branch is arranged between the objective lens 122 and the second imaging branch 38.

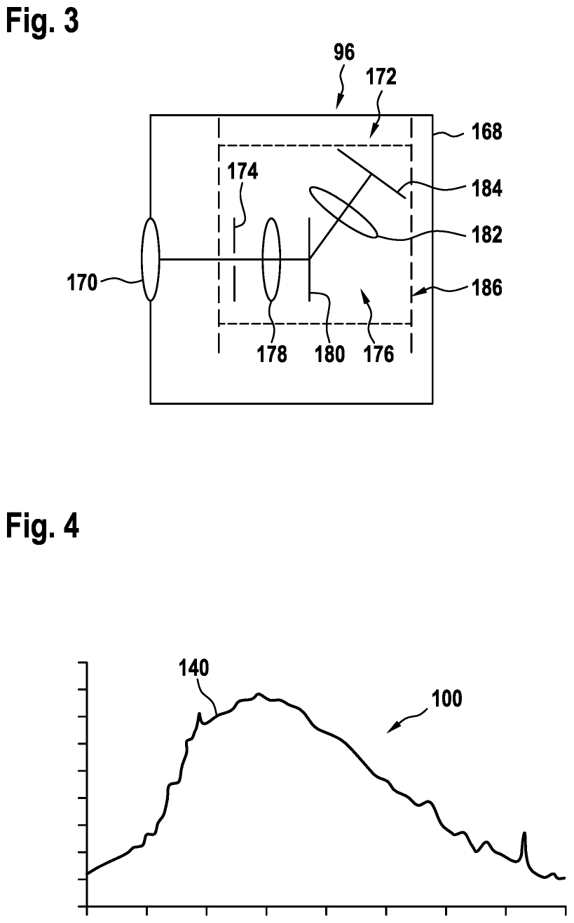

The second imaging branch 38 has at least one second spectrally selective beam splitter 40. In terms of its optical properties, the second spectrally selective beam splitter 40 is embodied differently from the first spectrally selective beam splitter 22. The second spectrally selective beam splitter 40 spectrally selectively splits the further first partial image representation 30 into at least one second spectral partial image representation 42 of a second spectral range 44 and at least one further second spectral partial image representation 46 of a further second spectral range 48. The second spectral range 44 is different from the further second spectral range 48 (cf. FIGS. 3, 4 and 5). The second spectrally selective beam splitter 40 here deflects the second spectral partial image representation 42, by 90° in the present case. The further second spectral partial image representation 46 passes through the second spectrally selective beam splitter 40 with a constant direction.

The second imaging branch 38 has at least one second image sensor 50. The second image sensor 50 is configured for capturing the second spectral partial image representation 42. As a result of the deflection of the second spectrally selective beam splitter 40, the second spectral partial image representation 42 is directed onto the second image sensor 50. In this case, the second image sensor lies in an image plane of the first spectral partial image representation 26. The second image sensor 50 has a sensor plane 82. The sensor plane 82 is arranged at least substantially parallel to the central axis 84 of the shaft 16.

For the purpose of relaying the further second partial image representation 46, the second imaging branch 38 has at least one second relay optical unit 52. The second relay optical unit 52 is disposed after the second selective beam splitter 40 upstream in terms of luminous flux. The second relay optical unit 52 has an object plane 56. Furthermore, the second relay optical unit 52 has an image plane 74. The first relay optical unit 36 and the second relay optical unit 52 are arranged one behind the other such that the image plane 54 of the first relay optical unit 36 is identical to the object plane 56 of the second relay optical unit 52. In this way, the second relay optical unit 52 relays the further second partial image representation 30 true to scale.

The second spectrally selective beam splitter 40 is arranged between the first relay optical unit 36 and the second relay optical unit 52 of the second imaging branch 38. To put it more precisely, the second spectrally selective beam splitter 40 is arranged between the first relay optical unit 36 and the image plane 54 of the first relay optical unit 36 or the object plane 56 of the second relay optical unit 52.

The second image sensor 50 is arranged between the first relay optical unit 36 and the second relay optical unit 52 of the second imaging branch 38. To put it more precisely, the second image sensor 50 is arranged between the first relay optical unit 36 and the image plane 54 of the first relay optical unit 36 or the object plane 56 of the second relay optical unit 52. The second image sensor 50 is disposed after the second spectrally selective beam splitter 40 upstream in terms of luminous flux. In this case, the second spectrally selective beam splitter 40 and the second image sensor 50 are arranged such that an optical distance through the second spectrally selective beam splitter 40 to the second image sensor 50 corresponds to an optical distance through the second spectrally selective beam splitter 40 to the image plane 54 of the first relay optical unit 36 or the object plane 56 of the second relay optical unit 52.

The second imaging branch 38 has at least one second further optical component 132. A refractive index and/or a material of the second further optical component 132 at least substantially corresponds to a refractive index and/or a material of the second spectrally selective beam splitter 40. The second further optical component 132 is arranged between the first relay optical unit 36 and the second relay optical unit 52. To put it more precisely, the second further optical component 132 is arranged between the second beam splitter 40 and the second relay optical unit 52. The further second optical component 132 is arranged in such a way that the image plane 54 of the first relay optical unit 36 or the object plane 56 of the second relay optical unit 52 lies between the second further optical component 132 and the second spectrally selective beam splitter 40. The second spectrally selective beam splitter 40 and the second further optical component are arranged mirror-symmetrically with respect to the mutually identical image plane 54 of the first relay optical unit 36 and object plane 56 of the second relay optical unit 52. In the present case, the second further optical component 132 is at least partly integrally embodied with or connected to the second beam splitter 40.

The imaging channel 18 has at least one third imaging branch 58. The third imaging branch 58 is arranged at least partly in the shaft 16. In the present case, the third imaging branch 58 is arranged completely in the shaft 16. The third imaging branch 58 is disposed after the second imaging branch 38 upstream in terms of luminous flux. In other words, the second imaging branch 38 is arranged between the first imaging branch 20 and the third imaging branch 58.

The third imaging branch 58 has at least one third spectrally selective beam splitter 60. In terms of its optical properties, the third spectrally selective beam splitter 60 is embodied differently from the first spectrally selective beam splitter 20. Furthermore, in terms of its optical properties, the third spectrally selective beam splitter 60 is embodied differently from the second spectrally selective beam splitter 40. The third spectrally selective beam splitter 60 spectrally selectively splits the further second partial image representation 46 into at least one third spectral partial image representation 62 of a third spectral range 64 and at least one further third spectral partial image representation 66 of a further third spectral range 68. The third spectral range 64 is different from the further third spectral range 68.

The third imaging branch 58 has at least one third image sensor 70. The third image sensor 70 is configured for capturing the third spectral partial image representation 62. As a result of the deflection of the second spectrally selective beam splitter 40, the third spectral partial image representation 62 is directed onto the third image sensor 70. In this case, the third image sensor 70 lies in an image plane of the third spectral partial image representation 62. The third image sensor has a sensor plane 82. The sensor plane 82 is arranged at least substantially parallel to the central axis 84 of the shaft 16.

For the purpose of relaying the further third partial image representation 66, the third imaging branch 58 has at least one third relay optical unit 72. The third relay optical unit 72 is disposed after the third selective beam splitter 60 upstream in terms of luminous flux. The third relay optical unit 72 has an object plane 76. Furthermore, the third relay optical unit 72 has an image plane 134. The third relay optical unit 72 and the second relay optical unit 52 are arranged one behind the other such that the image plane 74 of the second relay optical unit 52 is identical to the object plane 76 of the third relay optical unit 72. In this way, the third relay optical unit 72 relays the further third spectral partial image representation 66 true to scale. The third relay optical unit 72 has a rod lens pair 130.

The third spectrally selective beam splitter 60 is arranged between the second relay optical unit 52 and the third relay optical unit 72 of the third imaging branch 58. To put it more precisely, the third spectrally selective beam splitter 60 is arranged between the second relay optical unit 52 and the image plane 74 of the second relay optical unit 52 or the object plane 76 of the third relay optical unit 72.

The third image sensor 70 is arranged between the second relay optical unit 52 and the third relay optical unit 72 of the third imaging branch 58. To put it more precisely, the third image sensor 70 is arranged between the second relay optical unit 52 and the image plane 74 of the second relay optical unit 52 or the object plane 76 of the third relay optical unit 72. The third image sensor 70 is disposed after the third spectrally selective beam splitter 60 upstream in terms of luminous flux. In this case, the third spectrally selective beam splitter 60 and the third image sensor 70 are arranged such that an optical distance through the third spectrally selective beam splitter 60 to the third image sensor 70 corresponds identically to an optical distance through the third spectrally selective beam splitter to the image plane 74 of the second relay optical unit 52 or the object plane 76 of the third relay optical unit 72.

The third imaging branch 58 has at least one third further optical component 136. A refractive index and/or a material of the third further optical component 136 at least substantially corresponds to a refractive index and/or a material of the third spectrally selective beam splitter 60. The third further optical component 136 is arranged between the second relay optical unit 52 and the third relay optical unit 72. To put it more precisely, the third further optical component 136 is arranged between the third spectrally selective beam splitter 60 and the third relay optical unit 72. The third further optical component 136 is arranged in such a way that the image plane 74 of the second relay optical unit 52 or the object plane 76 of the third relay optical unit 72 lies between the third further optical component 136 and the third spectrally selective beam splitter 60. The third spectrally selective beam splitter 60 and the third further optical component 136 are arranged mirror-symmetrically with respect to the mutually identical image plane and object plane of the second relay optical unit 52 and of the third relay optical unit 72. In the present case, the second further optical component 132 is at least partly integrally embodied with or connected to the second beam splitter 40.

The imaging channel 18 has at least one printed circuit board 86. At least one of the image sensors 34, 50, 70 is arranged on the printed circuit board. In the present case, even all of the image sensors 34, 50, 70 are arranged on the printed circuit board 86. The printed circuit board 86 is embodied as a PCB. Alternatively, such a printed circuit board can also be a flexible printed circuit board or the like.

The imaging device 10 has at least one light source 98. The light source 98 is controlled by the control device 102. The light source 98 is arranged in a separate housing of the imaging device 10. The light source 98 emits illumination light in at least one operating state. The light source 98 has at least one luminous element 104. In the present case, the luminous element 104 is embodied as a white light lamp, in particular a xenon light source, which is provided with filters, for example, in order to reduce the light bands known for xenon and to generate a homogeneous white light distribution. In an operating state, the light source 98 illuminates the examination region to be imaged by means of the imaging device 10 with the illumination light. The illumination light has an illumination spectrum 100 (cf. FIG. 4). However, the luminous element can also be for example an LED, an OLED, a laser, in particular a diode laser, or the like. Moreover, the light source can have a plurality of luminous elements which can have different emissions characteristics. An illumination spectrum could be varied by way of individual control or switching on and/or off of such luminous elements. Alternatively, the luminous element could also be a spectrally tunable luminous element, such as a tunable laser, for example.

The imaging device 10 has at least one optical waveguide 116 (cf. FIG. 2). In the present case, the imaging device 10 has a plurality of optical waveguides 116. For the sake of clarity, only one optical waveguide 116 is provided with a reference sign in the drawings and described in greater detail in the description. The optical waveguide 116 is configured to guide the illumination light provided by the light source 98 along the shaft 16 from a proximal end to a distal end in order to illuminate the examination region. The optical waveguide 116 is arranged in the overshaft 106. The optical waveguide 116 is arranged in an intermediate region between the shaft 16 and the overshaft 106. The optical waveguide 116 has a plurality of optical fibers (not illustrated) in the present case. Individual optical fibers here could be individually connected to individual luminous elements of the light source. Alternatively, one or more luminous elements of the light source could be arranged at the distal end portion of the overshaft, as a result of which the optical waveguides could be dispensed with.

The imaging channel 18 has at least one camera 96, for capturing at least one of the partial image representations 26, 30, 42, 46, 62, 66 of the imaging branches 20, 38, 58. The camera 96 is arranged in a proximal, specifically in particular extracorporeal, end portion 121 of the shaft 16 of the imaging device 10. The camera 96 has a camera housing 168. Further components of the camera 96 are arranged in the camera housing 168. In the present case, the camera 96 is embodied as a hyperspectral camera. Alternatively, however, the camera could be embodied as a multispectral camera or white light camera.

FIG. 3 shows a set-up of the camera 96 in a schematic illustration. The camera 96 has at least one input objective lens 170. The input objective lens 170 is arranged in the camera housing 168. The input objective lens 170 is disposed after the imaging branches 20, 38, 58 upstream in terms of luminous flux. The input objective lens 170 is disposed after the third imaging branch 58. The input objective lens 170 is situated behind the third relay optical unit 72. The input objective lens 170 is configured to generate a partial image representation of the examination region to be examined in an image plane.

The camera 96 has a spectrometer 172. The spectrometer 172 is connected to the control device 102 for the purpose of control. The spectrometer 172 is arranged in the camera housing 168. The spectrometer 172 is arranged behind the input objective lens 170 upstream in terms of luminous flux.

The spectrometer 172 has at least one stop 174. The input objective lens 170 focusses 170 the image representation onto the stop 174. The stop 174 is arranged in an image plane of the image generated by the input objective lens 170. A distance between the input objective lens 170 and the stop 174 at least substantially corresponds to the image distance of the input objective lens 170. The stop 174 lies in the image plane. The stop 174 is configured to select a region of the image generated by the input objective lens 170. For this purpose, the stop 174 has an opening. The opening has the form of a slit. A main extension direction of the opening defines a first direction. This first direction is at least substantially parallel to the image plane of the image generated by the input objective lens 170. The stop 174 is configured to select a strip of the image which has a width of at least 15 μm and/or of at most 30 μm.

The spectrometer 172 has an internal optical unit 176. The internal optical unit 176 is arranged behind the stop 174 upstream in terms of luminous flux. The internal optical unit 176 has at least one internal lens 178. This internal lens 178 is arranged behind the stop 174 upstream in terms of luminous flux. A distance between the internal lens 178 and the stop 174 corresponds to the focal length of the internal lens 178. In this way, the internal lens 178 images the stop 174 into infinity.

Furthermore, the spectrometer 172 has at least one dispersive element 180. The dispersive element 180 is arranged behind the internal lens 178 upstream in terms of luminous flux. The dispersive element 180 is provided for wavelength-dependent fanning out of light. In the present case, the dispersive element 180 is configured so as to fan out this light in a second direction. The second direction is at least substantially perpendicular to the main extension of the opening of the stop 174. By way of example, the dispersive element 180 can be a prism. In the present case, the dispersive element 180 is embodied as an optical grating, in particular blazed grating.

The internal optical unit 176 has at least one further internal lens 182. The further objective lens 182 is arranged behind the dispersive element 180 upstream in terms of luminous flux. In this way, the dispersive element 180 is arranged between the internal lens 178 and the further internal lens 182. In other words, the dispersive element 180 is arranged within the internal optical unit 176. A distance between the further internal lens 182 and the dispersive element 180 corresponds to the focal length of the further internal lens 182. The further internal lens 182 is configured to sharply image the light fanned out by the dispersive element 180.

The spectrometer 172 has a camera image sensor 184. The camera image sensor 184 is connected to the control device 102. The camera image sensor 184 is arranged behind the further internal lens 182 upstream in terms of luminous flux. In other words, the further internal lens 182 is arranged between the dispersive element 180 and the camera image sensor 184. The camera image sensor 184 is a monochromatic sensor. Such a monochromatic sensor has only a single spectral sensitivity. The camera image sensor 184 is a two-dimensional CMOS camera image sensor. Alternatively, it could be a CCD camera image sensor.

The camera 96 has an adjusting device 186. The adjusting device 186 is connected to the control device 102 for the purpose of control. The adjusting device 186 is arranged in the camera housing 168. The adjusting device 186 is configured to adjust at least the stop 174 relative to the input objective lens 170. In the present case, the entire spectrometer 172 is adjusted relative to the input objective lens 170. The adjusting device 186 has at least one bearing. The bearing is configured for movable mounting of the spectrometer 172 relative to the input objective lens 170. In the present case, the bearing is embodied as a linear bearing. By way of example, the bearing can comprise guide rails arranged in a manner extending along the second direction. The adjusting device 186 furthermore has an adjusting actuator for drive purposes. In the present case, the adjusting actuator is embodied as a linear actuator. In order to be able to obtain uniform adjustment, for example, the adjusting actuator must be embodied as a piezoactuator.

Adjusting the stop 174 relative to the input objective lens 170 enables spectra to be recorded for different image segments of the examination region to be examined. By means of displacement, the entire examination region can thus be spectrally scanned, which makes it possible to generate an image representation including spectral information.

The camera 96 enables the examination region to be represented with higher spectral resolution. However, a frame rate as a result of this technique may be low relative to other methods. The camera 96 is used in a method for fine diagnostics. For example if color images recorded in a by means of image sensors of the imaging branches of the imaging channel have to be verified.

FIG. 4 shows a schematic diagram illustrating the spectral properties of the light source 98. The diagram shows an abscissa axis. A wavelength is plotted on the abscissa axis. Furthermore, the diagram shows an ordinate axis. An intensity is plotted on the ordinate axis. The diagram furthermore shows an illumination characteristic curve. The illumination characteristic curve 140 characterizes the illumination spectrum 100 of the light source 98. In the present case, the illumination characteristic curve 140 shows a profile which is characteristic of white light.

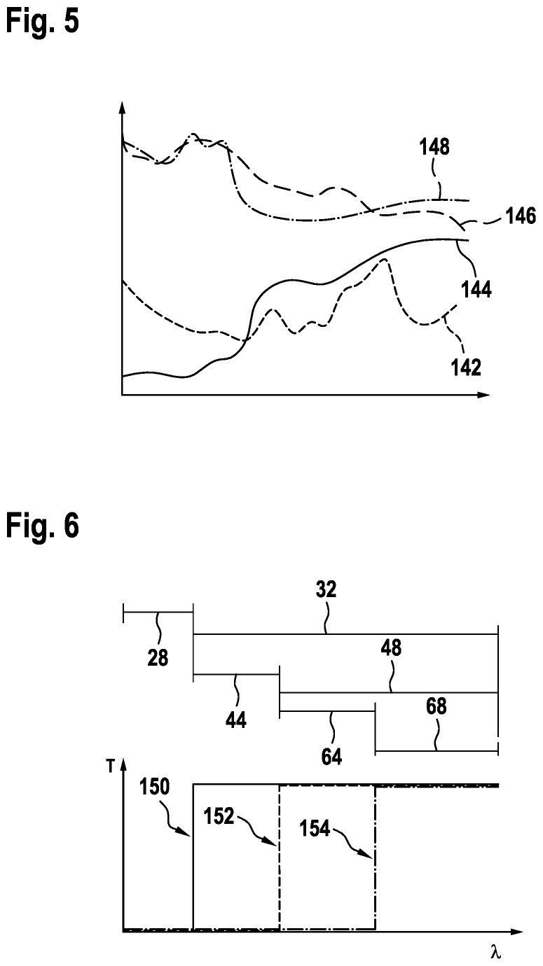

FIG. 5 shows a schematic diagram illustrating exemplary spectral properties of the examination region. The diagram shows an abscissa axis. A wavelength is plotted on the abscissa axis. Furthermore, the diagram shows an ordinate axis. An intensity is plotted on the ordinate axis.

The diagram shows a first spectral characteristic curve 142. The first spectral characteristic curve 142 characterizes the spectral properties of the examination region. In the present case, the first spectral characteristic curve 142 exhibits a profile which is characteristic of fatty tissue.

The diagram furthermore shows a second spectral characteristic curve 144. The second spectral characteristic curve 144 characterizes the spectral properties of the examination region. In the present case, the second spectral characteristic curve 144 exhibits a profile which is characteristic of aqueous tissue.

The diagram furthermore shows a third spectral characteristic curve 146. The third spectral characteristic curve 146 characterizes the spectral properties of the examination region. In the present case, the third spectral characteristic curve 146 exhibits a profile which is characteristic of deoxygenated tissue.

The diagram furthermore shows a fourth spectral characteristic curve 148. The fourth spectral characteristic curve 148 characterizes the spectral properties of the examination region. In the present case, the fourth spectral characteristic curve 148 exhibits a profile which is characteristic of oxygenated tissue.

The spectral properties of the tissue vary over the entire examination region. A tissue type and/or a tissue property of the examination region can thus be deduced on the basis of said properties. In the present case, the spectral lines 142, 144, 146, 148 are reflection spectra of the examination region. Alternatively, however, transmission spectra, fluorescence spectra and/or phosphorescence spectra could also be used. This may be dependent on an operating mode. Depending on the operating mode, a reflection, transmission, fluorescence and/or phosphorescence could be examined as a response of the examination region to the incident illumination light.

FIG. 6 shows a schematic diagram illustrating optical properties of the beam splitters 22, 40, 60 of the imaging branches 20, 38, 58 of the imaging channel 18 in an idealized manner (cf. FIG. 2). The diagram shows an abscissa axis. A wavelength is plotted on the abscissa axis. Furthermore, the diagram shows an ordinate axis. An intensity is plotted on the ordinate axis. A transmission intensity is plotted on the ordinate axis. The transmission intensity describes that proportion of the light incident on the beam splitter 22, 40, 60 which is transmitted by the beam splitters 22, 40, 60.

The diagram shows a first transmission characteristic curve 150. The first transmission characteristic curve 150 describes the spectral transmission behavior of the first spectrally selective beam splitter 22. The first transmission characteristic curve 150 characterizes the spectral splitting of the image representation of the examination region into the first spectral partial image representation 26 and the further first spectral partial image representation 30 by the first spectrally selective beam splitter 22. The first transmission characteristic curve 150 characterizes the spectral splitting of an original spectral response of the examination region to the illumination light into the first spectral range 28 and the further first spectral range 32 by the first spectrally selective beam splitter 22. In the first spectral range 28, the transmission proportion is at most 25%. Even at most 5% in the present case. In the further first spectral range 32, the transmission proportion is at least 75%. Even at least 95% in the present case. The first spectral range 28 and the further first spectral range 32 are separated from one another by a slope of the transmission characteristic curve. The slope rises from the first spectral range 28 as far as the further first spectral range 32. The slope has a slope inflection point. The slope inflection point is at at least substantially 540 nm. However, it is also conceivable that the spectral ranges could be interchanged with one another. Moreover, the transmission characteristic curve could define a plurality of spectral ranges which could be spectrally offset with respect to one another.

The diagram shows a second transmission characteristic curve 152. The second transmission characteristic curve 152 describes the spectral transmission behavior of the second spectrally selective beam splitter 40. The second transmission characteristic curve 152 characterizes the spectral splitting of the further first partial image representation 30 into the second spectral partial image representation 42 and the further second spectral partial image representation 46 by the second spectrally selective beam splitter 40. The second transmission characteristic curve 152 characterizes the spectral splitting of the further first spectral range 32 into the second spectral range 44 and the further second spectral range 48 by the second spectrally selective beam splitter 40. In the second spectral range 44, the transmission proportion is at most 25%. Even at most 5% in the present case. In the further second spectral range 48, the transmission proportion is at least 75%. Even at least 95% in the present case. The second spectral range 44 and the further second spectral range 48 are separated from one another by a slope of the transmission characteristic curve 152. The slope rises from the second spectral range 44 as far as the further second spectral range 48. The slope has a slope inflection point. The slope inflection point is at at least substantially 680 nm. However, it is also conceivable that the spectral ranges could be interchanged with one another. Moreover, the transmission characteristic curve could define a plurality of spectral ranges which could be spectrally offset with respect to one another.

The diagram shows a third transmission characteristic curve 154. The third transmission characteristic curve 154 describes the spectral transmission behavior of the third spectrally selective beam splitter 60. The third transmission characteristic curve 154 characterizes the spectral splitting of the further second partial image representation 46 into the third spectral partial image representation 62 and the further third spectral partial image representation 66 by the third spectrally selective beam splitter 60. The third transmission characteristic curve 154 characterizes the spectral splitting of the further second spectral range 48 into the third spectral range 64 and the further third spectral range 68 by the third spectrally selective beam splitter 60. In the third spectral range 64, the transmission proportion is at most 25%. Even at most 5% in the present case. In the further third spectral range 68, the transmission proportion is at least 75%. Even at least 95% in the present case. The third spectral range 64 and the further third spectral range 68 are separated from one another by a slope of the third transmission characteristic curve 154. The slope rises from the third spectral range 64 as far as the further third spectral range 68. The slope has a slope inflection point. The slope inflection point is at at least substantially 670 nm. However, it is also conceivable that the spectral ranges could be interchanged with one another. Moreover, the transmission characteristic curve could define a plurality of spectral ranges which could be spectrally offset with respect to one another.

FIG. 7 shows a schematic diagram illustrating further optical properties of the beam splitters 22, 40, 60 of the imaging branches 20, 38, 58 of the imaging channel 18 in an idealized manner. The diagram shows an abscissa axis. A wavelength is plotted on the abscissa axis. Furthermore, the diagram shows an ordinate axis. An intensity is plotted on the ordinate axis. In the case of FIG. 7, a reflection intensity is plotted on the ordinate axis. The reflection proportion describes that proportion of light incident on the beam splitter 22, 40, 60 which is reflected by the beam splitter 22, 40, 60. Apart from an absorption proportion that is approximately negligible, the reflection behavior of the beam splitters 22, 40, 60 is substantially complementary to the transmission behavior described above.

The diagram shows a first reflection characteristic curve 156. The first reflection characteristic curve 156 describes the spectral reflection behavior of the first spectrally selective beam splitter 22. The first reflection characteristic curve 156 characterizes the spectral splitting of the image representation of the examination region into the first spectral partial image representation 26 and the further first spectral partial image representation 30 by the first spectrally selective beam splitter 22. The first reflection characteristic curve 156 characterizes the spectral splitting of an original spectral response of the examination region to the illumination light into the first spectral range 28 and the further first spectral range 32 by the first spectrally selective beam splitter 22. In the first spectral range 28, the reflection proportion is at least 75%. Even at least 95% in the present case. In the further first spectral range 32, the reflection proportion is at most 25%. Even at most 5% in the present case. The first spectral range 28 and the further first spectral range 32 are separated from one another by a slope of the reflection characteristic curve 156. The slope falls from the first spectral range 28 as far as the further first spectral range 32. The slope has a slope inflection point. The slope inflection point is at at least substantially 540 nm. However, it is also conceivable that the spectral ranges could be interchanged with one another. Moreover, the transmission characteristic curve could define a plurality of spectral ranges which could be spectrally offset with respect to one another.

The diagram shows a second reflection characteristic curve 158. The second reflection characteristic curve 158 describes the spectral reflection behavior of the second spectrally selective beam splitter 40. The second reflection characteristic curve 158 characterizes the spectral splitting of the further first partial image representation 30 into the second spectral partial image representation 42 and the further second spectral partial image representation 46 by the second spectrally selective beam splitter 40. The second reflection characteristic curve 158 characterizes the spectral splitting of the further first spectral range 32 into the second spectral range 44 and the further second spectral range 48 by the second spectrally selective beam splitter 40. In the second spectral range 44, the reflection proportion is at least 75%. Even at least 95% in the present case. In the further second spectral range 48, the reflection proportion is at most 25%. Even at most 5% in the present case. The second spectral range 44 and the further second spectral range 48 are separated from one another by a slope of the second reflection characteristic curve 158. The slope falls from the second spectral range 44 as far as the further second spectral range 48. The slope has a slope inflection point. The slope inflection point is at at least substantially 680 nm. However, it is also conceivable that the spectral ranges could be interchanged with one another. Moreover, the transmission characteristic curve could define a plurality of spectral ranges which could be spectrally offset with respect to one another.

The diagram shows a third reflection characteristic curve 160. The third reflection characteristic curve 160 describes the spectral reflection behavior of the third spectrally selective beam splitter 60. The third reflection characteristic curve 160 characterizes the spectral splitting of the further second partial image representation 46 into the third spectral partial image representation 62 and the further third spectral partial image representation 66 by the third spectrally selective beam splitter 60. The third reflection characteristic curve 160 characterizes the spectral splitting of the further second spectral range 48 into the third spectral range 64 and the further third spectral range 68 by the third spectrally selective beam splitter 60. In the third spectral range 64, the reflection proportion is at least 75%. Even at least 95% in the present case. In the further third spectral range 68, the reflection proportion is at most 25%. Even at most 5% in the present case. The third spectral range 64 and the further third spectral range 68 are separated from one another by a slope of the third transmission characteristic curve 154. The slope falls from the third spectral range 64 as far as the further third spectral range 68. The slope has a slope inflection point. The slope inflection point is at at least substantially 670 nm. However, it is also conceivable that the spectral ranges could be interchanged with one another. Moreover, the transmission characteristic curve could define a plurality of spectral ranges which could be spectrally offset with respect to one another.

In this way, the beam splitters 20, 44, 60 subdivide light of the image representation into respective partial image representations 22, 30, 42, 46, 62, 66 and their respective partial spectral ranges.

FIG. 8 illustrates detection properties of the image sensors 34, 50, 70 with the aid of a diagram. In the present case, the image sensors 34, 50, 70 are embodied substantially identically to one another. Alternatively, the image sensors could be embodied at least partly differently from one another and have different detection properties, for example.

The diagram in FIG. 8 shows an abscissa axis. A wavelength is plotted on the abscissa axis. Furthermore, the diagram shows an ordinate axis. An intensity is plotted on the ordinate axis. In the case of FIG. 8, a signal intensity is plotted on the ordinate axis. Said signal intensity describes the signal strength with which a sensor senses light of a certain wavelength. The properties of the image sensor 34, 50, 70 are described in greater detail below with reference to one of the image sensors 34, 70. This description is applicable to the other image sensors 34, 50, 70.

The first image sensor 34 has at least one first spectral sensitivity 88. The diagram shows a first sensitivity characteristic curve 162, which characterizes the first spectral sensitivity 88. The first spectral sensitivity 88 corresponds to a blue spectral band of an RGB sensor.

Furthermore, the first image sensor 34 has at least one second spectral sensitivity 90. The diagram shows a second sensitivity characteristic curve 164, which characterizes the second spectral sensitivity 90. The second spectral sensitivity 90 corresponds to a green spectral band of an RGB sensor.

Furthermore, the first image sensor 34 has at least one third spectral sensitivity 92. The diagram shows a third sensitivity characteristic curve 166, which characterizes the third spectral sensitivity 92. The third spectral sensitivity 92 corresponds to a red spectral band of an RGB sensor.

Alternatively, the spectral sensitivities could also show a different spectral behavior. By way of example, this could represent ultraviolet, near-infrared, infrared spectral bands of a sensor.

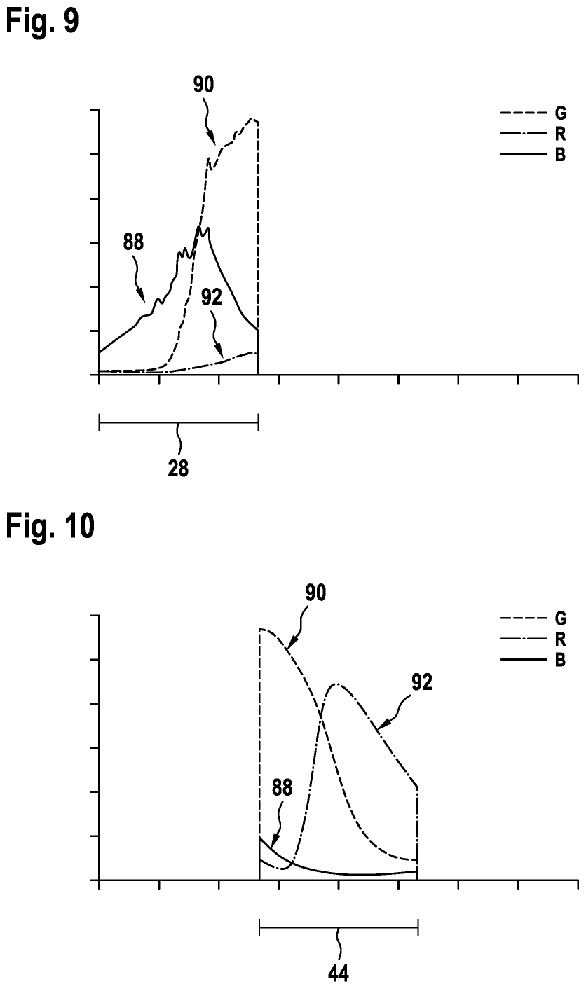

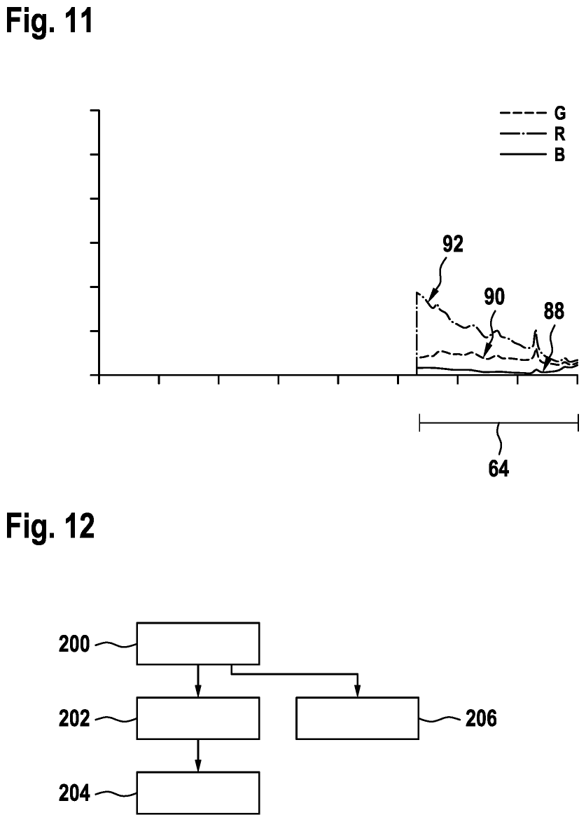

FIGS. 9, 10, 11 illustrate the spectral sensitivities 88, 90, 92 of the image sensors 34, 50, 70 within the partial spectral ranges assigned to the respective sensors. Comparison of FIG. 4 with FIGS. 9, 10, 11 reveals that the illumination spectrum 100 comprises a spectral range which is broader than at least one spectral range of the partial image representations 22, 30, 42, 46, 62, 66 of the imaging branches 20, 38, 58. In the present case, the illumination spectrum 100 is broader than the first spectral range 28, the second spectral range 44 and the third spectral range 64. During a measurement, the superposition of the spectral response of the examination region with the spectral ranges 28, 44, 64 within the respective partial spectral range yields three support points in each case, which are used for an analysis of the type and/or property of the examination region.

FIG. 12 shows a schematic flow chart of an exemplary method for operating the endoscopic imaging device 10. This method can directly also be applied to operation of an exoscopic device.

The method comprises at least a method step 200. In the method step 200, the endoscopic device is oriented toward an examination region. The light source 98 is activated. The light impinges on the examination region. The light is at least partly reflected by the examination region. Furthermore, the light is at least partly absorbed and/or transmitted by the examination region. Furthermore, the examination region can emit fluorescence or phosphorescence as a response to the light. The light that is reflected or emitted by the examination region is captured by means of the endoscopic device. In this case, the light passes into the imaging channel 18. An image representation of the examination region is generated in the imaging channel. Said image representation is split into the partial image representations 22, 30, 42, 46, 62, 66 of respective partial spectral ranges by the imaging branches 20, 38, 58. The partial image representations 22, 30, 42, 46, 62, 66 of respective partial spectral ranges are captured by the respective assigned image sensors 34, 50, 70. The image sensors 34, 50, 70 output support points according to their spectral sensitivities 88, 90, 92. The signals of the image sensors 34, 50, 70 are communicated to the control device.

The method comprises a further method step 202. In the further method step 202, the control device 120 compares the support points with comparison values stored in the memory, for example with curve profiles which are characteristic of different tissue types and/or properties, as illustrated in FIG. 5. On the basis of the support points, the control device 102 then assigns these support points to the different tissue types and/or properties. This is carried out for each pixel or each RGB pixel group of a respective image sensor 34, 50, 70.

The method comprises a further method step 204. In the method step 204, the control device 102 stores the assignment in a memory or outputs this by means of a color coded image. In this case, for each assignment of the tissue property and/or type, an individual image can be output, or in a common color coded image. The individual images can be represented on the display simultaneously, individually or in particular alternately. Furthermore, the color images are recorded, generated and/or represented with a frame rate of at least 15 frames per second. A video is thus generated which is constituted from the color images and advantageously represents the examination region in real time.

The method comprises at least a further method step 206. In the method step 206, the control device 102 generates a white light image of the examination region by way of superposition of the partial image representations 22, 30, 42, 46, 62, 66. This examination region can be displayed separately from the individual image or color coded image of the assignment of the tissue property and/or type. Alternatively or additionally, however, it is conceivable to output them jointly in a manner superposed with one another in a superposition image. Furthermore, the white light image, individual images and/or the common color coded image could be displayed alternately.

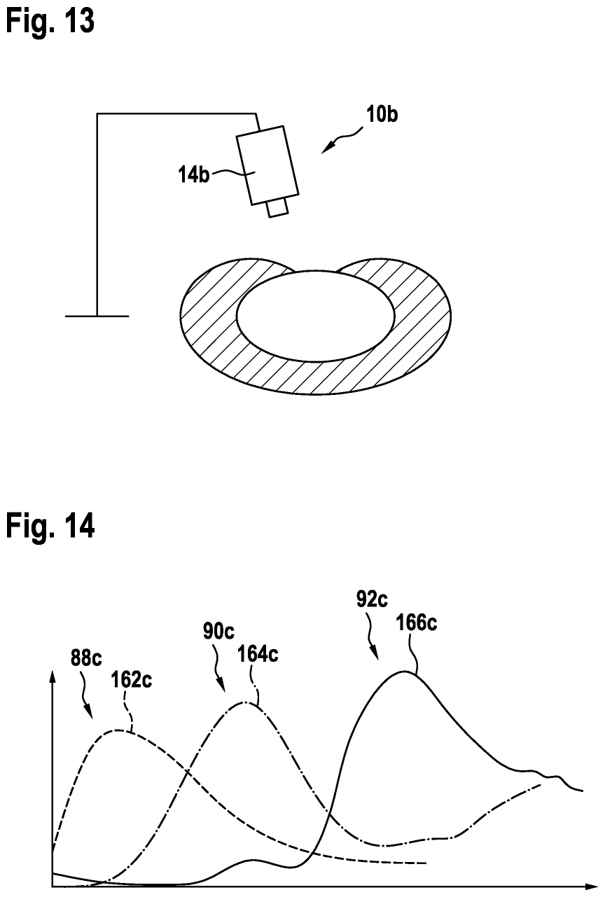

FIGS. 13 to 16 show further exemplary embodiments of the disclosure. The following descriptions and the drawings are limited substantially to the differences between the exemplary embodiments, and with regard to identically designated components, in particular in regard to components having identical reference signs, in principle reference is also made to the drawings and/or the description of the other exemplary embodiments, in particular in FIGS. 1 to 12. In order to differentiate the exemplary embodiments, the letter a has been placed after the reference signs of the exemplary embodiment in FIGS. 1 to 12. In the exemplary embodiments in FIGS. 13 to 18, the letter b, c, d, e, f and g has been appended to a respective reference sign for differentiation from the previous exemplary embodiment.

FIG. 13 shows a schematic illustration of an exoscopic imaging device 10b in a perspective view. The present embodiment differs from the previous embodiment substantially in terms of an embodiment of the shaft 16b. In the case of an exoscopic device, said shaft is not configured to be inserted into a cavity. The exoscopic imaging device 10b completely forms an exoscope 14b. Alternatively, the exoscopic imaging device could form only a functional constituent part of an exoscope. By means of such an exoscopic embodiment, even from outside a cavity it is possible to record multispectral and/or hyperspectral images in an examination region of the cavity.

FIG. 14 shows optical properties of an alternative imaging device 10c. The present imaging device 10c differs from the previous imaging devices substantially in terms of image sensors 34c, 50c, 70c embodied differently from one another.

In the present case, the imaging device 10c has at least one imaging channel 18c. The imaging channel 18c comprises at least three imaging channels. In the present case, the imaging channels have different image sensors 34c, 50c, The image sensors 34c, 50c, 70c have different spectral sensitivities 88c, 90c, 92c. FIG. 12 shows a diagram similar to that in FIG. 7. In the present case, however, the image sensors 34c, 50c, 70c have different spectral sensitivities 88c, 90c, 92c. Each sensor even has respectively only one spectral sensitivity 88c, 90c, 92c. As a result, it is possible to avoid a superposition of the spectral sensitivities 88c, 90c, 92c with regard to different partial spectral ranges. However, it is also conceivable for the image sensors to have a plurality of spectral sensitivities 88c, 90c, 92c which are all different from one another.

The first image sensor 34c has at least one spectral sensitivity 88c. The diagram shows a first sensitivity characteristic curve 162c, which characterizes the first spectral sensitivity 88c. The first spectral sensitivity 88c corresponds to a blue spectral band of an RGB sensor.

The second image sensor 50c has at least one spectral sensitivity 90c. The diagram shows a second sensitivity characteristic curve 164c, which characterizes the second spectral sensitivity 90c. The second spectral sensitivity 90c corresponds to a green spectral band of an RGB sensor.

The third image sensor 70c has at least one spectral sensitivity 92c. The diagram shows a third sensitivity characteristic curve 166c, which characterizes the third spectral sensitivity 92c. The third spectral sensitivity 92 corresponds to a red spectral band of an RGB sensor.

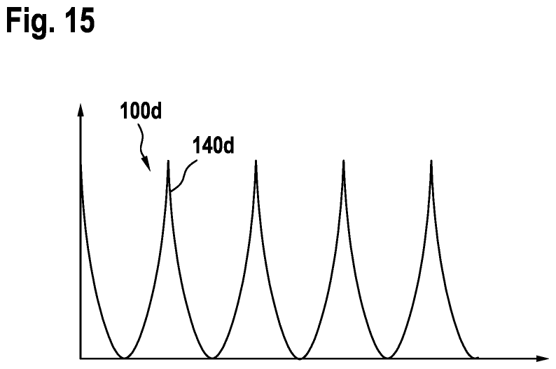

FIG. 15 shows optical properties of a further embodiment of a further endoscopic and/or exoscopic imaging device 10d. The present exemplary embodiment differs from the previous exemplary embodiment substantially in terms of an embodiment of the light source 98d.

The light source 98d emits illumination light comprising at least one illumination spectrum 100d which lies within at least one spectral range 28d, 32d, 44d, 48d, 64d, 68d of partial image representations 22d, 30d, 42d, 46d, 62d, 66d of imaging branches 20d, 38d, 58d of an imaging channel 18d of the imaging device (cf. FIGS. 5, 6). The illumination spectrum 100d has a width which is smaller than the spectral range 28d, 32d, 44d, 48d, 64d, 68d of the partial image representation within which the latter lies. The light source can comprise for example as a light element one or more LEDs, which can be operated by themselves, jointly or alternately.

FIG. 15 shows a schematic diagram illustrating the spectral properties of the light source 98. The diagram shows an abscissa axis. A wavelength is plotted on the abscissa axis. Furthermore, the diagram shows an ordinate axis. An intensity is plotted on the ordinate axis. The diagram furthermore shows an illumination characteristic curve 140d of the light source 98d. The illumination characteristic curve 140d comprises characterizes the illumination spectrum 100d of the light source 98d. In the present case, the illumination characteristic curve 140d shows a profile which is characteristic of an LED. The illumination characteristic curve 140d has various primary maxima which are used for example for a spectral analysis of fatty tissue, a water content, oxygenation, deoxygenation or the like.

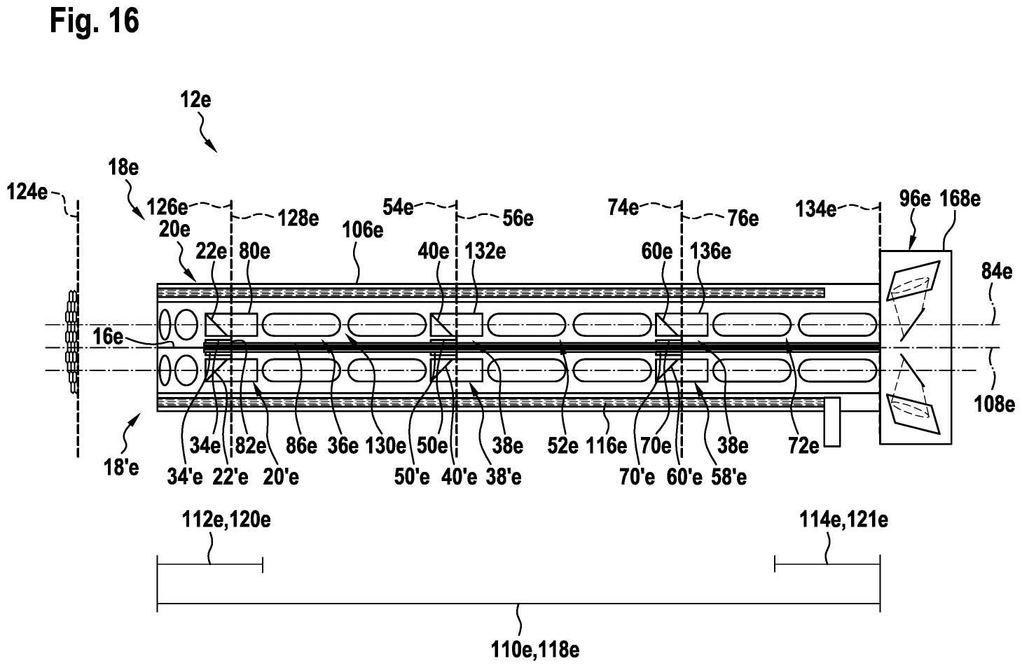

FIG. 16 shows a schematic illustration of an alternative endoscopic and/or exoscopic imaging device 10e in a sectional view. The present imaging device 10e differs from the previous imaging device substantially in terms of a number of imaging channels 18e, 18′e.

In the present case, the imaging device 10e has at least one imaging channel 18e. Furthermore, the imaging device 10e has at least one further imaging channel 18′e. Overall, the imaging device 10e has two imaging channels 18e, 18′e. Identical reference signs are used below for the components of the imaging channels. For differentiation, an apostrophe has been placed after the reference signs of the components of the further imaging channel 18′e. The use of two imaging channels 18e, 18′e enables advantageous stereoscopic multi- and/or hyperspectral imaging. Moreover, a spectral resolution can be increased further by the use of a plurality of imaging channels. In order to further improve and/or increase a spatial representation and/or spectral resolution, it is conceivable that the imaging device can have additional imaging channels.

The imaging channel 18e and the further imaging channel 18′e are arranged offset with respect to one another. The imaging channels 18e, 18′e are arranged offset laterally with respect to one another. The imaging channels 18e, 18′e are arranged offset with respect to one another substantially perpendicularly to central axes of the respective accommodating shafts of the imaging channels 18e, 18′e. The central axes of the imaging channels 18e, 18′e are at least substantially parallel to one another. Viewing directions of the imaging channels 18e, 18′e are at least substantially parallel. Alternatively, however, the central axes of the imaging channels could also be arranged at an angle with respect to one another. Viewing directions of the imaging channels could also be at an angle with respect to one another.

The imaging channel 18e and the further imaging channel 18′e are arranged mirror-symmetrically with respect to one another. The imaging device 10e has an overshaft 106e. The imaging channels 18e, 18′e are arranged jointly in an overshaft 106e. The overshaft 106e comprises a central axis 108e. The imaging channels 18e, 18′e are arranged mirror-symmetrically with respect to said central axis 108e.

The imaging channels 18e, 18′e each have at least one imaging branch 38e, 58e. In the present case, the imaging channels each have three imaging branches 20e, 38e, 58e. The imaging channels 18e, 18′e are embodied at least partly differently from one another. The imaging channels differ at least in terms of a spectral selectivity of at least one spectrally selective beam splitter 22e, 40e, 60e, 22′e, 40′e, 60′e of their imaging branches 20e, 38e, 58e, 20′e, 38′e, 58′e. In the present case, the spectral selectivities of all further spectrally selective beam splitters 22′e, 40′e, 60′e of the further imaging channel 18′e are different from the spectral selectivities of all the spectrally selective beam splitters 22e, 40e, 60e of the imaging channel 18e. Furthermore, the imaging channels differ at least in terms of a spectral sensitivity 88e, 90e, 92e of at least one image sensor 34e, 50e, 70e of their imaging branches 20e, 38e, 58e, 20′e, 38′e, 58′e. In the present case, the spectral sensitivities 88e, 90e, 92e of all further image sensors 34′e, 50′e, 70′e of the further imaging channel 18′e are different from the spectral sensitivities 88e, 92e of all the spectrally selective beam splitters 22e, 40e, 60e, of the imaging channel 18e.

In regard to a method, this embodiment affords the advantage that different tissue properties and/or types can be assigned by means of the different imaging branches 20e, 38e, 58e, 20′e, 38′e, 58′e. In order to obtain a uniform representation, the different images can be matched e.g. by way of feature tracking, as a result of which the complete information is also available for a stereo image.

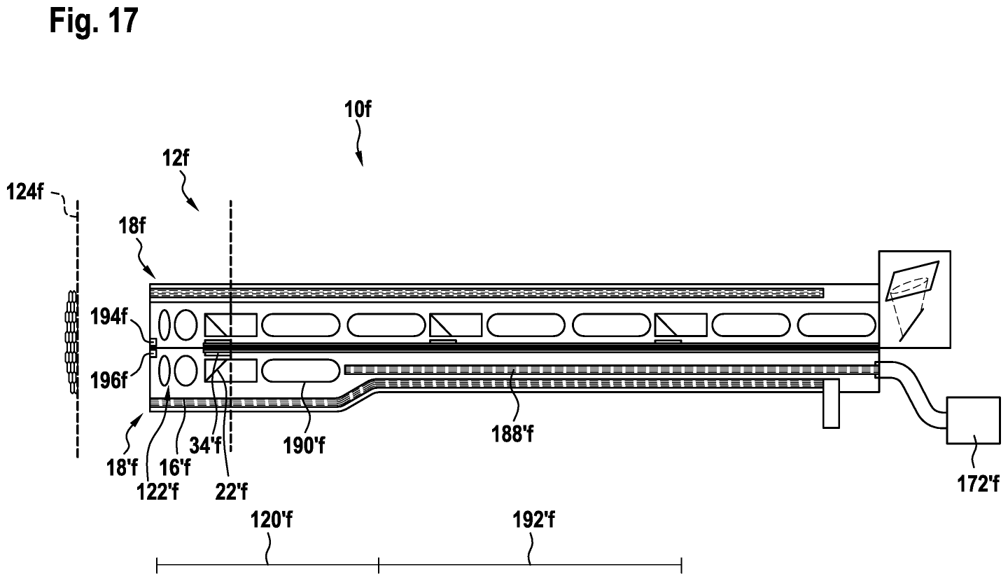

FIG. 17 shows a schematic illustration of a further alternative endoscopic and/or exoscopic imaging device 10f in a sectional view. The present imaging device differs from the previous imaging device substantially in terms of an embodiment of the further imaging channel 18′f of the imaging device.

The imaging channel 18f is configured for multispectral and/or hyperspectral imaging. In contrast thereto, the further imaging channel 18′f is configured for white light imaging. By virtue of the fact that the imaging channels 18f, 181 are embodied differently from one another, e.g. by way of a different number of beam splitter, are coordinated with one another during a joint representation of image representations recorded by means of the imaging channels by means of said image representations being amplified digitally, for example, and/or brightnesses and/or contrasts being adapted.

The further imaging channel 18′f has an objective lens 122′f. The objective lens 122′f is arranged at least partly in the shaft 16′f. The objective lens 122′f is arranged in the distal end portion 120′f of the shaft 16′f. The objective lens 122′f is configured for generating an optical image representation of the examination region. The objective lens 122′f has an object plane 124′f. In an operating state, the examination region is situated in the object plane 1241. The objective lens 122′f furthermore has an image plane. The objective lens 122′f images an optical image representation of the examination region situated in the object plane onto the image plane.

The further imaging channel 181 has a first imaging branch 201. The further first imaging branch 20′f is arranged partly in the shaft 16′. In the present case, the further first imaging branch 201 is arranged completely in the shaft 16′f. The further first imaging branch 201 is disposed after the objective lens 1221 upstream in terms of luminous flux.

The further first imaging branch 20′f has at least one first spectrally selective beam splitter 22′f. The first spectrally selective beam splitter 22′f splits the optical image representation of the examination region into at least one first spectral partial image representation of a first spectral range and at least one further first spectral partial image representation of a further first spectral range 321. The first spectrally selective beam splitter 221 deflects the first spectral partial image representation, by 90° in the present case. The further first spectral partial image representation passes through the first spectrally selective beam splitter 221 in a constant direction. The first spectral partial image representation extends over a visible spectral range. The further first spectral partial image representation extends over the near infrared range.

The further first imaging branch 201 has at least one further first image sensor 341. The further first image sensor 34′f is configured for capturing the first spectral partial image representation. As a result of the deflection of the first spectrally selective beam splitter 221, the first spectral partial image representation is directed onto the first image sensor. The first image sensor 34′f lies in an image plane of the first spectral partial image representation. Consequently, the first spectral partial image representation is sharply imaged onto the first image sensor 341.

The further first imaging branch 20′f does not have a relay optical unit, however, in the present case. Consequently, the further imaging channel 18′f is also free of a relay optical unit.

Instead, the imaging channel 181 has an optical fiber 1881. The optical fiber 1881 is arranged in the shaft 161. For the purpose of focusing the partial image representation onto the optical fiber 1881, the further first imaging branch 20′f comprises a focus lens 1901. The focus lens 190′f is arranged between beam splitter 221 and optical fiber 188′f.

Furthermore, the imaging device 10′f comprises a spectrometer 1721. The spectrometer 172′f is configured to record an overall spectrum of the further first partial image representation. The spectrometer 1721 is connected to the optical fiber.

The imaging device 101 has a control device. The control device is configured to match at least one multispectral and/or hyperspectral image recorded by means of the imaging channel 18f with at least one white light image recorded by the further imaging channel 18′f. For this purpose, the control device uses a matching algorithm. In the present case, feature tracking is used, but some other algorithm can also be used. By virtue of the matching, it is possible to generate both white light and multispectral and/or hyperspectral stereo images or stereo videos, even though in each case only one of the imaging channels 18f, 18′f is configured for white light imaging or multispectral and/or hyperspectral imaging.

The imaging device 10′f furthermore comprises at least one temperature sensor 194f. The temperature sensor 194f is arranged in the distal end portion 120′f of the shaft 16′f. The temperature sensor 194f is configured for temperature-dependent control of at least the imaging channels 18f, 18′f. By means of targeted activation and/or deactivation of the electronics, such as the camera image sensors, for example, a limit temperature can be prevented from being exceeded. Furthermore, an irradiation time to be complied with for a light source of the imaging device 10f is also regulated by means of the limit temperature to be complied with.

The imaging device 10′f has at least one motion sensor 196f. The motion sensor is arranged in a distal end portion 120′f of the shaft 16′f. The motion sensor 196f is configured for motion-dependent control of at least the imaging channels 18f, 181. The motion sensor 196f is configured to register a constant position of the imaging device 10′f during a recording of an image representation. For the case where a motion is registered, a message is transferred to the control device and a recording is stopped. Furthermore, it is possible to record a motion by means of the motion sensor 196f and to correct imaging on the basis of this captured motion.

The shaft 16′f has a distal end portion 120′f, which is widened in relation to a central portion 192′f. Both the imaging channel 18′f and the further imaging channel 18′f are arranged in this distal end portion 120′f. However, the central portion 1921 is substantially filled by the imaging channel 18′f.

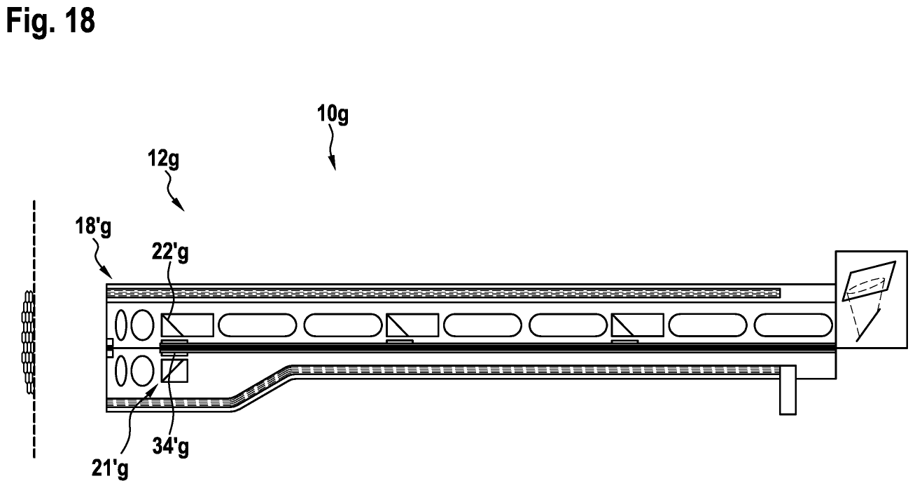

FIG. 18 shows a schematic illustration of an additional alternative endoscopic and/or exoscopic imaging device 10g in a sectional view. The present imaging device 10g differs from the previous imaging device substantially in terms of an embodiment of the further imaging channel 18′g of the imaging device 10′g.

In the present case, the further imaging channel 18′g has a first imaging strand 21′g instead of a first imaging branch. The imaging strand 21′g is embodied substantially equivalently to one of the imaging branches described above, this having a mirror instead of a beam splitter, said mirror being configured to direct light onto an image sensor of the imaging strand 21g. The imaging strand 21′f is furthermore free of a relay optical unit. Furthermore, the imaging strand 121′f is free of branches of an optical path.

1. A surgical imaging device for spectral imaging, in particular multispectral imaging and/or hyperspectral, imaging for an, the surgical imaging device including at least one shaft; at least one imaging channel arranged at least partly in the shaft the imaging channel having at least one first imaging branch which includes at least one first spectrally selective beam splitter which spectrally selectively splits an optical image representation of an original spectral range into at least one first spectral partial image representation of a first spectral range and at least one further first spectral partial image representation of a further first spectral range, wherein the first spectral range is different from the further first spectral range; wherein the first imaging branch further includes a first image sensor, at least for capturing the first spectral partial image representation-PO, and at least one first relay optical unit for relaying the further first partial image representation, the imaging device comprising:
at least one second spectrally selective beam splitter disposed within the at least one second imagining branch of the imaging channel, the at least second spectrally selective beam splitter spectrally selectively splits the further first partial image representation of the further first spectral range into at least one second spectral partial image representation of a second spectral range and at least one further second spectral partial image representation of a further second spectral range, wherein the second spectral range is different from the further second spectral range and;
wherein the second imaging branch further includes at least one second image sensor for capturing the second spectral partial image representation, and at least one second relay optical unit, for relaying the further second partial image representation, wherein the first relay optical unit of the first imaging branch and the second relay optical unit of the second imaging branch are arranged one behind the other such that an image plane of the first relay optical unit is identical to an object plane of the second relay optical unit.

2. The surgical imaging device as set forth in claim 1, characterized in that the imaging channel has at least one third imaging branch which comprises at least one third spectrally selective beam splitter which spectrally selectively splits the further second spectral partial image representation of the further second spectral range into at least one third spectral partial image representation of a third spectral range and at least one further third spectral partial image representation of a further third spectral range, wherein the third spectral range is different from the further third spectral range and the third imaging branch includes at least one third image sensor, at least for capturing the third spectral partial image representation, and at least one third relay optical unit, for relaying the further third spectral partial image representation, wherein the second relay optical unit of the second imaging branch and the third relay optical unit of the third imaging branch are arranged one behind the other such that an image plane of the second relay optical unit is identical to an object plane of the third relay optical unit.

3. The surgical imaging device as set forth in claim 1, characterized in that at least one spectrally selective beam splitter and/or at least one image sensor of the corresponding first, second and third imaging branches is arranged between two of the first, second and third relay optical units of different first, second and third imaging branches.

4. The surgical imaging device as set forth in claim 1, characterized in that at least one of the first, second and third imaging branches includes at least one further optical component, the refractive index of which and/or the material of which at least substantially correspond(s) to a refractive index and/or a material of a first, second and third spectrally selective beam splitters of the respective first, second and third imaging branches, and is arranged between two relay optical units of different first, second and third imaging branches.

5. The surgical imaging device as set forth in claim 3, characterized in that the first, second and third spectrally selective beam splitters and the further optical component are arranged mirror-symmetrically with respect to the mutually identical image plane and object plane of two of the corresponding first, second and third relay optical units of different first, second and third imaging branches.

6. The surgical imaging device at least as set forth in claim 3, characterized in that the first, second and third spectrally selective beam splitters and the further optical component are embodied or connected to one another at least partly integrally.

7. The surgical imaging device as set forth in claim 1, characterized in that at least one of the spectral ranges of the partial image representations of the first, second and third imaging branches comprises shorter wavelengths than at least one other spectral range of the partial image representations of the corresponding first, second and third imaging branches.

8. The surgical imaging device as set forth in claim 1, characterized in that at least one of the first, second and third image sensors or at least two of the first, second and third image sensors of the first, second and third imaging branches has/have a sensor plane arranged at least substantially parallel to a central axis of the shaft.

9. The surgical imaging device as set forth in any of the claim 1, characterized in that the imaging channel has at least one printed circuit board on which at least one of the first, second and third image sensors or at least two of the first, second and third image sensors of the first, second and third imaging branches is/are arranged.

10. The surgical imaging device as set forth in claim 1, characterized in that at least one of the first, second and third image sensors of the first, second and third imaging branches has at least one spectral sensitivity which is different from a spectral sensitivity of another image sensor of the imaging branches.

11. The surgical imaging device as set forth in claim 1, characterized in that at least one of the first, second and third image sensors or at least two of the first, second and third image sensors of the first, second and third imaging branches has/have at least one first spectral sensitivity and at least one second spectral sensitivity, and in particular at least one third spectral sensitivity, depending on which of said sensor(s) capture(s) a partial image representation of the first, second and third imaging branches.

12. The surgical imaging device as set forth in claim 1, characterized in that the imaging channel has at least one camera arranged at a proximal and in particular extracorporeal end portion of the shaft, for capturing at least one of the partial image representations of the imaging branches.

13. The surgical imaging device as set forth in claim 12, characterized in that the camera is a multispectral and/or hyperspectral camera.

14. The surgical imaging device as set forth in claim 1, further including at least one light source which illuminates an examination region to be imaged with at least one illumination spectrum in at least one operating state.

15. The surgical imaging device as claimed in claim 14, characterized in that the at least one light source emits illumination light comprising an illumination spectrum which is broader than at least one spectral range of the partial image representations of the imaging branches.

16. The surgical imaging device as claimed in claim 14, characterized in that the at least one light source emits illumination light comprising at least one illumination spectrum which lies within at least one spectral range of the partial image representations of the first, second and third imaging branches.

17. The surgical imaging device as set forth in claim 1, further including at least one further imaging channel.

18. The surgical imaging device as claimed in claim 17, characterized in that the imaging channel and the at least one further imaging channel differ from one another at least in terms of a spectral sensitivity of at least one spectrally selective beam splitter of their corresponding first, second and third imaging branches and/or in terms of a spectral sensitivity of a corresponding at least one first, second and third image sensor of a respective first, second and third imaging branches.

19. The surgical imaging device as claimed in claim 17, characterized in that the imaging channel and the at least one further imaging channel are arranged mirror-symmetrically with respect to one another.

20. The surgical imaging device as set forth claim 17, wherein the at least one further imaging channel is free of the relay optical unit.

21. The surgical imaging device as set forth in claim 20, characterized in that the shaft is substantially filled by the imaging channel at least in portions.

22. The surgical imaging device as claimed in claim 20, characterized in that the imaging channel is configured for multispectral and/or hyperspectral imaging and the further imaging channel is configured for white light imaging.

23. The surgical imaging device as claimed in claim 22, including a control device configured to match at least one multispectral and/or hyperspectral image recorded by the imaging channel with at least one white light image recorded by the further imaging channel.

24. The surgical imaging device as set forth in claim 1, further including at least one temperature sensor is arranged in a distal end portion of the shaft, said at least one temperature sensor being configured for temperature-dependent control of the at least the imaging channel.

25. The surgical imaging device as set forth in claim 1, further including at least one motion sensor is arranged in a distal end portion of the shaft, said at least one motion sensor being configured for motion-dependent control of at least the imaging channel.

26. A medical imagining scope including the imaging device set forth in claim 1.

27. The medical imaging scope as set forth in claim 26, wherein the medical imaging scope is one of an endoscope and an exoscope.

28. A method of operating the surgical imaging device as set forth in claim 1 comprising the steps of:
providing a light source;
illuminating an area of interest with the light source; and
generating a spectral image from at least the first spectral partial image representation captured by the first image sensor and/or the further first partial spectral image representation captured by the further first image sensor.
